SEQUENCE LISTING (6 sequences)

SEQ ID NO: 1 - DNA, 7031 bp, Homo sapiens
ttcaccggaaaacacagtagctgtagctgcacatagacgcttcctctttctgtttccccctcatccccctccctagacctctctgggctttgacgtcatgtgtgctcctttcggttgccatagcaaccccattccccaagccctctgtccgtctcctctggtaggttccacaatggtacaggcagcatcacgctgcacaatggtttccaggcagtgaaagagggtgattcagcaagccactcttcttctattttctttaacctccccttcactttttatttttatgggggtgggtggtgcttgctatatgcttacctttttcttttcttttttcatttttacaaatttccttttttgtcctcacccctcaattcctaggggcttgagtgagtttaagattgggttttcttggaaatcacctgtccatcgttaattttaaacaatctccatatctccaaagaatctcttccatgttagtctggaatgtggttaatgaaaaacaagtagggaggatttctggggcaaacactgccggatcaggatcgtagttctcaggcacggaatggctagtgtgagaaacaccaacagcaggcccatctcagatcttcactatggcaacttatgcaagaaactgttgaattagacccgtttcctatagatgagaaaccatacaagctgtggtatttatgagcctccatttcttatactactgcagtgaaccaacattggatgtgaaaattgccttttgtcagggattcgataaacaagtggatgtgtcatatattgccaaacattacaacatgagcaaaagcaaagttgacaaccagttctacagtgtggaagtgggagactcaaccttcacagttctcaagcgctaccagaatctaaagcctattggctctggggctcagggcatagtttgtgccgcgtatgatgctgtccttgacagaaatgtggccattaagaagctcagcagaccctttcagaaccaaacacatgccaagagagcgtaccgggagctggtcctcatgaagtgtgtgaaccataaaaacattattagtttattaaatgtcttcacaccccagaaaacgctggaggagttccaagatgtttacttagtaatggaactgatggatgccaacttatgtcaagtgattcagatggaattagaccatgagcgaatgtcttacctgctgtaccaaatgttgtgtggcattaagcacctccattctgctggaattattcacagggatttaaaaccaagtaacattgtagtcaagtctgattgcacattgaaaatcctggactttggactggccaggacagcaggcacaagcttcatgatgactccatatgtggtgacacgttattacagagcccctgaggtcatcctggggatgggctacaaggagaacgtggatatatggtctgtgggatgcattatgggagaaatggttcgccacaaaatcctctttccaggaagggactatattgaccagtggaataaggtaattgaacaactaggaacaccatgtccagaattcatgaagaaattgcaacccacagtaagaaactatgtggagaatcggcccaagtatgcgggactcaccttccccaaactcttcccagattccctcttcccagcggactccgagcacaataaactcaaagccagccaagccagggacttgttgtcaaagatgctagtgattgacccagcaaaaagaatatcagtggacgacgccttacagcatccctacatcaacgtctggtatgacccagccgaagtggaggcgcctccacctcagatatatgacaagcagttggatgaaagagaacacacaattgaagaatggaaagaacttatctacaaggaagtaatgaattcagaagaaaagactaaaaatggtgtagtaaaaggacagccttctccttcaggtgcagcagtgaacagcagtgagagtctccctccatcctcgtctgtcaatgacatctcctccatgtccaccgaccagaccctggcatctgacactgacagcagcctggaagcctcggcaggacccctgggttgttgcaggtgactagccgcctgcctgcgaaacccagcgttcttcaggagatgatgtgatggaacacacacacacgcagacacacacacacacacaaatgcagacacacaacatcaagaaaacagcaagggagagaatccaagcctaaaattaaataaatctttcagcctgcttcttccccagggttctgtattgcagctaagctcaaatgtatatttaacttctagttgctcttgctttggtcttcttccaatgatgcttactacagaaagcaaatcagacacaattagagaagccttttccataaagtgtaattttaatggctgcaaaaccggcaacctgtaactgcccttttaaatggcatgacaaggtgtgcagtggccccatccagcatgtgtgtgtctctatcttgcatctacctgctccttggcctagtcagatggatgtagatacagatccgcatgtgtctgtattcatacagcactacttacttagagatgctactgtcagtgtcctcagggctctaccaagacataatgcactggggtaccacatggtccatttcatgtgatctattactctgacataaacccatctgtaatatattgccagtatataagctgtttagtttgttaattgattaaactgtatgtcttataagaaaacatgtaaagggggaatatatggggggagtgagctctctcagacccttgaagatgtagcttccaaatttgaatggattaaatggcacctgtataccaatttgtagaaagaacatatgtgatacttatgtaaagtgtggggcgagtgggtaacagttttcaggcacaaaatggtttggccttctgaaggcaggtgtgaataaaagctgagagtcttttctgtgacagtaaagagtaggagagactggagggtagtgtgtggtgtggactaagactgaaaggaggttattacccagatactaggagacactggtgtccctgttttagagatttggctctatacccatttgtcatttctcaattactggtcacaacaagaggccactggagaaagtggcaggtggtgaagcaaggttattgtgtgtgcttttgatgataagaatctctctctggaactgagcagaattgcctccatgtgaattccttcaacaaaaaaatgagcctgcctagactactttcagattgtggattgtgttttccatgtgtgggctgcttccctgattcatactctgccaaagttttattttgagaaataatattactttcctcttctgaaataaaataataataataataatgaaacccccaaaccacagtgtgagtctcaggttagcatttgaaaacatctccagagacattgttattcctcaggagtttccctgactccttaatgtggctgatgtttcatgtttatttatttattttaataagtatgagcaatcgaagggctgatcatctgagttttgtactgcagtagatgtaagagcaataactctgcccataaatttactgctgccaaatagttttagcaatatgagtttgtcttaaaagccatgaagcattaatgtcaatgtttagcaaaatctccagaatttagcttaaattaaattaaatgaaagtgctttcctgagcacttaaggaggcactgtgaaacatcttggttatgtacaaggcaataccaaggtgatctcagcaataatcaggcttcccttcttattcacttcgaagtatccatcacagttatttgcactctgaccatgttggtgctcactagagtggtgatatttcacctgaagaagtgaaagacaggaagtggatggttgatgagtgtgaactgattggcaaatagaatatccccaaaacagagcaaacaagaatgccagtgctctgaaaactgccttaaatataataatagggaactttctttctctcttcaagcaatagagaccacagggaactgcacccacctgcaccatcgccatgccagtggtgctgccattctgagattatttcacacagtcagattcagtacacgcagtcatttgtttaaaataaatagaagaacattcctttgtgcagcttgtcccctggaataaaaatggcccattttctctattttgctaccattttttttctggttaatattgcagataacctgattgttaaaaatgcctcctcagtcacttgagctccctctctttcatattcctgttttttttttttttttttaaaaacttctcctctctgtatctctaattctttactgattttctttctcggtacttttctcttgatatatctgtggcttctcccctcccttactttataccacatgggattaagtgcggagagcaggaagctgctttaaagagagtctcaaggggaatttcatcagccgcaagatgacatcacaagacagaaggaagtctccagccagcatcttcaaagtgcttgacaggctatctctcgtacctgcataagtggtgaatttccccctcagagtaaaccaggcaagaactagacagggcccctcccccgctgttagtaccttcccaaagctcaattagtcttacatcttctgtgcatggctcttgggagctggttagctttagccagatttcccttatggacaagagaaaaatggaacaaattccttgaatataagtatcatttacaaatagaggtaaggatttggcccccagaatttttgcaggatcagttttattaggtgattaaagcaaggcagatgatggaacaacaaagagaatcaccactgtaaaagccccccagtcctttgcccactaggtctgctttcctataatatgtctaggaggtgatgatattatcaaattcacagaatataatatgttctgcattcacttgtctgtcttaacacacacacacacacacacacacacacacacacacacacaccccaaatatttcttaaatagtcacctgttatttcccaagaacatcccaacttttccggtaggcaagtgtcaagtcacctggacaatattcctgaaaaaaagtgaaggttctacctggggttggggctcattttggaacatgaagttggcactctgaggagcctaaccttctgttaggcttcctttggggcagttggtttgcagtgatccttaataaaatagtttgcagtattttgtaggaaggatggctgtgtactcatgtgcagggaaaattaaactgctgcaagagtttggcaaaaccgccaccactttactattaatatgtctttactatttttatatgtgttttgtgggtcagcgtgttcaattttaaataggaatacactagccttacaacggaatttcttcttgtggaataaaaactgataataacctttcatcttcaagaatctgatgactgagttttgtaggaatgtacccaacagttggaccagagcctggccagttcccaccaattggagtaagctatttaattgagggttaagaagctcgatagttttttcaatctgttatcagaaaaatattatctaaagacactgatagcagctgactggtaaaactgaaagttggccttagtaaatggaactagtaaaaaatagataaaaatatgtaaaaccttttatgtcctgtcaaggaaaaaaaaataagcaaacttgatttgttttaaaattgtaaatattcattgcaaaacctaaattatacaaaatctatttcgatttgaatctcttaacatatgaaaagccacatcttaagtgcattatgctctgtaaggcatgttattagccagctttgctgaaaaatgtattcttctacagctcagaaagaggagtaaaattgctctagaaatttttgatctcagccactttgcatccaaagaggaacccaactccccactcacaatcccccacacacattttaattataagagttcaacattttccacagatatcattaagccaagcaaggaaactcctctctgtaaaaacaacaaaaaagttgcaatgtaaatagttattttagctattttctttgtgattagaggataagattttgttatttcttttggtacttagtcctttaactatctgaaaatggctcactttgacctgtttctttggtcagccaaactgaagtgaggggcctctgtttatatatagaatcttgtatatgtaactttacttatgtgggaccaatggccagtatattctaatggacacaaatctcccaatccttatgtaggattatctgttcaccttgccatgtccatcttgcaatgagacctaacttgcacctgcctgtttttctgcatgattggactccaaagactattaccaatgatgagaattgtcttgtgtcagtgtttataaaatggacttaaagcacacacacatgcatacccacattcacatgcacccttacagttctagatcaaccctggaaggcaaaaccatgtacgtccagtttctttgcaaggtctgagcagtgagaactaaattcagtcagtctaatgggtaaaatgcaacctcagtgatgcttaataaggatttgaaatcgttcaagtcatttctgtgttctttctcacaccctttcctttctattttgtcttcttttattttcaaaatgtctgtggaacaagaggatgaatcgggggagaacttctgcttcttttgactctttttaggtaatatggagtcaatatattatgatttctgtttcttatggattattaaaatcagaggaaatctgcttctctcaacataaaaccaaatccaaacccagacattgttggagtttgcctgttgacaatggttatgatgctgctcgttgtcttgagttgcatgtaacgacagtagctctgaagggtgtcatgatttcattttggcaactggtaaaagaaacttgtagtgtggtctgattgtgccagggaataaataagaaataaggaatgaaaaaaaaaaaaaaaaaaa

SEQ ID NO: 2 - DNA, 7031 bp, Homo sapiens; identical to SEQ ID NO: 1

SEQ ID NO: 3 - DNA, 6572 bp, Homo sapiens
agaaatggcgtggcaggggacccagcgagcccagagggattttgccgctgcttcctctacccctgtatttcacgcagctctctaaattgactcagctccaggctagtgtgagaaacaccaacagcaggcccatctcagatcttcactatggcaacttatgcaagaaactgttgaattagacccgtttcctatagatgagaaaccatacaagctgtggtatttatgagcctccatttcttatactactgcagtgaaccaacattggatgtgaaaattgccttttgtcagggattcgataaacaagtggatgtgtcatatattgccaaacattacaacatgagcaaaagcaaagttgacaaccagttctacagtgtggaagtgggagactcaaccttcacagttctcaagcgctaccagaatctaaagcctattggctctggggctcagggcatagtttgtgccgcgtatgatgctgtccttgacagaaatgtggccattaagaagctcagcagaccctttcagaaccaaacacatgccaagagagcgtaccgggagctggtcctcatgaagtgtgtgaaccataaaaacattattagtttattaaatgtcttcacaccccagaaaacgctggaggagttccaagatgtttacttagtaatggaactgatggatgccaacttatgtcaagtgattcagatggaattagaccatgagcgaatgtcttacctgctgtaccaaatgttgtgtggcattaagcacctccattctgctggaattattcacagggatttaaaaccaagtaacattgtagtcaagtctgattgcacattgaaaatcctggactttggactggccaggacagcaggcacaagcttcatgatgactccatatgtggtgacacgttattacagagcccctgaggtcatcctggggatgggctacaaggagaacgtggatatatggtctgtgggatgcattatgggagaaatggttcgccacaaaatcctctttccaggaagggactatattgaccagtggaataaggtaattgaacaactaggaacaccatgtccagaattcatgaagaaattgcaacccacagtaagaaactatgtggagaatcggcccaagtatgcgggactcaccttccccaaactcttcccagattccctcttcccagcggactccgagcacaataaactcaaagccagccaagccagggacttgttgtcaaagatgctagtgattgacccagcaaaaagaatatcagtggacgacgccttacagcatccctacatcaacgtctggtatgacccagccgaagtggaggcgcctccacctcagatatatgacaagcagttggatgaaagagaacacacaattgaagaatggaaagaacttatctacaaggaagtaatgaattcagaagaaaagactaaaaatggtgtagtaaaaggacagccttctccttcagcacaggtgcagcagtgaacagcagtgagagtctccctccatcctcgtctgtcaatgacatctcctccatgtccaccgaccagaccctggcatctgacactgacagcagcctggaagcctcggcaggacccctgggttgttgcaggtgactagccgcctgcctgcgaaacccagcgttcttcaggagatgatgtgatggaacacacacacacgcagacacacacacacacacaaatgcagacacacaacatcaagaaaacagcaagggagagaatccaagcctaaaattaaataaatctttcagcctgcttcttccccagggttctgtattgcagctaagctcaaatgtatatttaacttctagttgctcttgctttggtcttcttccaatgatgcttactacagaaagcaaatcagacacaattagagaagccttttccataaagtgtaattttaatggctgcaaaaccggcaacctgtaactgcccttttaaatggcatgacaaggtgtgcagtggccccatccagcatgtgtgtgtctctatcttgcatctacctgctccttggcctagtcagatggatgtagatacagatccgcatgtgtctgtattcatacagcactacttacttagagatgctactgtcagtgtcctcagggctctaccaagacataatgcactggggtaccacatggtccatttcatgtgatctattactctgacataaacccatctgtaatatattgccagtatataagctgtttagtttgttaattgattaaactgtatgtcttataagaaaacatgtaaagggggaatatatggggggagtgagctctctcagacccttgaagatgtagcttccaaatttgaatggattaaatggcacctgtataccaatttgtagaaagaacatatgtgatacttatgtaaagtgtggggcgagtgggtaacagttttcaggcacaaaatggtttggccttctgaaggcaggtgtgaataaaagctgagagtcttttctgtgacagtaaagagtaggagagactggagggtagtgtgtggtgtggactaagactgaaaggaggttattacccagatactaggagacactggtgtccctgttttagagatttggctctatacccatttgtcatttctcaattactggtcacaacaagaggccactggagaaagtggcaggtggtgaagcaaggttattgtgtgtgcttttgatgataagaatctctctctggaactgagcagaattgcctccatgtgaattccttcaacaaaaaaatgagcctgcctagactactttcagattgtggattgtgttttccatgtgtgggctgcttccctgattcatactctgccaaagttttattttgagaaataatattactttcctcttctgaaataaaataataataataataatgaaacccccaaaccacagtgtgagtctcaggttagcatttgaaaacatctccagagacattgttattcctcaggagtttccctgactccttaatgtggctgatgtttcatgtttatttatttattttaataagtatgagcaatcgaagggctgatcatctgagttttgtactgcagtagatgtaagagcaataactctgcccataaatttactgctgccaaatagttttagcaatatgagtttgtcttaaaagccatgaagcattaatgtcaatgtttagcaaaatctccagaatttagcttaaattaaattaaatgaaagtgctttcctgagcacttaaggaggcactgtgaaacatcttggttatgtacaaggcaataccaaggtgatctcagcaataatcaggcttcccttcttattcacttcgaagtatccatcacagttatttgcactctgaccatgttggtgctcactagagtggtgatatttcacctgaagaagtgaaagacaggaagtggatggttgatgagtgtgaactgattggcaaatagaatatccccaaaacagagcaaacaagaatgccagtgctctgaaaactgccttaaatataataatagggaactttctttctctcttcaagcaatagagaccacagggaactgcacccacctgcaccatcgccatgccagtggtgctgccattctgagattatttcacacagtcagattcagtacacgcagtcatttgtttaaaataaatagaagaacattcctttgtgcagcttgtcccctggaataaaaatggcccattttctctattttgctaccattttttttctggttaatattgcagataacctgattgttaaaaatgcctcctcagtcacttgagctccctctctttcatattcctgttttttttttttttttttaaaaacttctcctctctgtatctctaattctttactgattttctttctcggtacttttctcttgatatatctgtggcttctcccctcccttactttataccacatgggattaagtgcggagagcaggaagctgctttaaagagagtctcaaggggaatttcatcagccgcaagatgacatcacaagacagaaggaagtctccagccagcatcttcaaagtgcttgacaggctatctctcgtacctgcataagtggtgaatttccccctcagagtaaaccaggcaagaactagacagggcccctcccccgctgttagtaccttcccaaagctcaattagtcttacatcttctgtgcatggctcttgggagctggttagctttagccagatttcccttatggacaagagaaaaatggaacaaattccttgaatataagtatcatttacaaatagaggtaaggatttggcccccagaatttttgcaggatcagttttattaggtgattaaagcaaggcagatgatggaacaacaaagagaatcaccactgtaaaagccccccagtcctttgcccactaggtctgctttcctataatatgtctaggaggtgatgatattatcaaattcacagaatataatatgttctgcattcacttgtctgtcttaacacacacacacacacacacacacacacacacacacacacaccccaaatatttcttaaatagtcacctgttatttcccaagaacatcccaacttttccggtaggcaagtgtcaagtcacctggacaatattcctgaaaaaaagtgaaggttctacctggggttggggctcattttggaacatgaagttggcactctgaggagcctaaccttctgttaggcttcctttggggcagttggtttgcagtgatccttaataaaatagtttgcagtattttgtaggaaggatggctgtgtactcatgtgcagggaaaattaaactgctgcaagagtttggcaaaaccgccaccactttactattaatatgtctttactatttttatatgtgttttgtgggtcagcgtgttcaattttaaataggaatacactagccttacaacggaatttcttcttgtggaataaaaactgataataacctttcatcttcaagaatctgatgactgagttttgtaggaatgtacccaacagttggaccagagcctggccagttcccaccaattggagtaagctatttaattgagggttaagaagctcgatagttttttcaatctgttatcagaaaaatattatctaaagacactgatagcagctgactggtaaaactgaaagttggccttagtaaatggaactagtaaaaaatagataaaaatatgtaaaaccttttatgtcctgtcaaggaaaaaaaaataagcaaacttgatttgttttaaaattgtaaatattcattgcaaaacctaaattatacaaaatctatttcgatttgaatctcttaacatatgaaaagccacatcttaagtgcattatgctctgtaaggcatgttattagccagctttgctgaaaaatgtattcttctacagctcagaaagaggagtaaaattgctctagaaatttttgatctcagccactttgcatccaaagaggaacccaactccccactcacaatcccccacacacattttaattataagagttcaacattttccacagatatcattaagccaagcaaggaaactcctctctgtaaaaacaacaaaaaagttgcaatgtaaatagttattttagctattttctttgtgattagaggataagattttgttatttcttttggtacttagtcctttaactatctgaaaatggctcactttgacctgtttctttggtcagccaaactgaagtgaggggcctctgtttatatatagaatcttgtatatgtaactttacttatgtgggaccaatggccagtatattctaatggacacaaatctcccaatccttatgtaggattatctgttcaccttgccatgtccatcttgcaatgagacctaacttgcacctgcctgtttttctgcatgattggactccaaagactattaccaatgatgagaattgtcttgtgtcagtgtttataaaatggacttaaagcacacacacatgcatacccacattcacatgcacccttacagttctagatcaaccctggaaggcaaaaccatgtacgtccagtttctttgcaaggtctgagcagtgagaactaaattcagtcagtctaatgggtaaaatgcaacctcagtgatgcttaataaggatttgaaatcgttcaagtcatttctgtgttctttctcacaccctttcctttctattttgtcttcttttattttcaaaatgtctgtggaacaagaggatgaatcgggggagaacttctgcttcttttgactctttttaggtaatatggagtcaatatattatgatttctgtttcttatggattattaaaatcagaggaaatctgcttctctcaacataaaaccaaatccaaacccagacattgttggagtttgcctgttgacaatggttatgatgctgctcgttgtcttgagttgcatgtaacgacagtagctctgaagggtgtcatgatttcattttggcaactggtaaaagaaacttgtagtgtggtctgattgtgccagggaataaataagaaataaggaatgaaaaaaaaaaaaaaaaaaa

SEQ ID NO: 4 - DNA, 7058 bp, Homo sapiens
ttcaccggaaaacacagtagctgtagctgcacatagacgcttcctctttctgtttccccctcatccccctccctagacctctctgggctttgacgtcatgtgtgctcctttcggttgccatagcaaccccattccccaagccctctgtccgtctcctctggtaggttccacaatggtacaggcagcatcacgctgcacaatggtttccaggcagtgaaagagggtgattcagcaagccactcttcttctattttctttaacctccccttcactttttatttttatgggggtgggtggtgcttgctatatgcttacctttttcttttcttttttcatttttacaaatttccttttttgtcctcacccctcaattcctaggggcttgagtgagtttaagattgggttttcttggaaatcacctgtccatcgttaattttaaacaatctccatatctccaaagaatctcttccatgttagtctggaatgtggttaatgaaaaacaagtagggaggatttctggggcaaacactgccggatcaggatcgtagttctcaggcacggaatggctagtgtgagaaacaccaacagcaggcccatctcagatcttcactatggcaacttatgcaagaaactgttgaattagacccgtttcctatagatgagaaaccatacaagctgtggtatttatgagcctccatttcttatactactgcagtgaaccaacattggatgtgaaaattgccttttgtcaggtgtgtgttccttacaggtaaaacaagggattcgataaacaagtggatgtgtcatatattgccaaacattacaacatgagcaaaagcaaagttgacaaccagttctacagtgtggaagtgggagactcaaccttcacagttctcaagcgctaccagaatctaaagcctattggctctggggctcagggcatagtttgtgccgcgtatgatgctgtccttgacagaaatgtggccattaagaagctcagcagaccctttcagaaccaaacacatgccaagagagcgtaccgggagctggtcctcatgaagtgtgtgaaccataaaaacattattagtttattaaatgtcttcacaccccagaaaacgctggaggagttccaagatgtttacttagtaatggaactgatggatgccaacttatgtcaagtgattcagatggaattagaccatgagcgaatgtcttacctgctgtaccaaatgttgtgtggcattaagcacctccattctgctggaattattcacagggatttaaaaccaagtaacattgtagtcaagtctgattgcacattgaaaatcctggactttggactggccaggacagcaggcacaagcttcatgatgactccatatgtggtgacacgttattacagagcccctgaggtcatcctggggatgggctacaaggagaacgtggatatatggtctgtgggatgcattatgggagaaatggttcgccacaaaatcctctttccaggaagggactatattgaccagtggaataaggtaattgaacaactaggaacaccatgtccagaattcatgaagaaattgcaacccacagtaagaaactatgtggagaatcggcccaagtatgcgggactcaccttccccaaactcttcccagattccctcttcccagcggactccgagcacaataaactcaaagccagccaagccagggacttgttgtcaaagatgctagtgattgacccagcaaaaagaatatcagtggacgacgccttacagcatccctacatcaacgtctggtatgacccagccgaagtggaggcgcctccacctcagatatatgacaagcagttggatgaaagagaacacacaattgaagaatggaaagaacttatctacaaggaagtaatgaattcagaagaaaagactaaaaatggtgtagtaaaaggacagccttctccttcaggtgcagcagtgaacagcagtgagagtctccctccatcctcgtctgtcaatgacatctcctccatgtccaccgaccagaccctggcatctgacactgacagcagcctggaagcctcggcaggacccctgggttgttgcaggtgactagccgcctgcctgcgaaacccagcgttcttcaggagatgatgtgatggaacacacacacacgcagacacacacacacacacaaatgcagacacacaacatcaagaaaacagcaagggagagaatccaagcctaaaattaaataaatctttcagcctgcttcttccccagggttctgtattgcagctaagctcaaatgtatatttaacttctagttgctcttgctttggtcttcttccaatgatgcttactacagaaagcaaatcagacacaattagagaagccttttccataaagtgtaattttaatggctgcaaaaccggcaacctgtaactgcccttttaaatggcatgacaaggtgtgcagtggccccatccagcatgtgtgtgtctctatcttgcatctacctgctccttggcctagtcagatggatgtagatacagatccgcatgtgtctgtattcatacagcactacttacttagagatgctactgtcagtgtcctcagggctctaccaagacataatgcactggggtaccacatggtccatttcatgtgatctattactctgacataaacccatctgtaatatattgccagtatataagctgtttagtttgttaattgattaaactgtatgtcttataagaaaacatgtaaagggggaatatatggggggagtgagctctctcagacccttgaagatgtagcttccaaatttgaatggattaaatggcacctgtataccaatttgtagaaagaacatatgtgatacttatgtaaagtgtggggcgagtgggtaacagttttcaggcacaaaatggtttggccttctgaaggcaggtgtgaataaaagctgagagtcttttctgtgacagtaaagagtaggagagactggagggtagtgtgtggtgtggactaagactgaaaggaggttattacccagatactaggagacactggtgtccctgttttagagatttggctctatacccatttgtcatttctcaattactggtcacaacaagaggccactggagaaagtggcaggtggtgaagcaaggttattgtgtgtgcttttgatgataagaatctctctctggaactgagcagaattgcctccatgtgaattccttcaacaaaaaaatgagcctgcctagactactttcagattgtggattgtgttttccatgtgtgggctgcttccctgattcatactctgccaaagttttattttgagaaataatattactttcctcttctgaaataaaataataataataataatgaaacccccaaaccacagtgtgagtctcaggttagcatttgaaaacatctccagagacattgttattcctcaggagtttccctgactccttaatgtggctgatgtttcatgtttatttatttattttaataagtatgagcaatcgaagggctgatcatctgagttttgtactgcagtagatgtaagagcaataactctgcccataaatttactgctgccaaatagttttagcaatatgagtttgtcttaaaagccatgaagcattaatgtcaatgtttagcaaaatctccagaatttagcttaaattaaattaaatgaaagtgctttcctgagcacttaaggaggcactgtgaaacatcttggttatgtacaaggcaataccaaggtgatctcagcaataatcaggcttcccttcttattcacttcgaagtatccatcacagttatttgcactctgaccatgttggtgctcactagagtggtgatatttcacctgaagaagtgaaagacaggaagtggatggttgatgagtgtgaactgattggcaaatagaatatccccaaaacagagcaaacaagaatgccagtgctctgaaaactgccttaaatataataatagggaactttctttctctcttcaagcaatagagaccacagggaactgcacccacctgcaccatcgccatgccagtggtgctgccattctgagattatttcacacagtcagattcagtacacgcagtcatttgtttaaaataaatagaagaacattcctttgtgcagcttgtcccctggaataaaaatggcccattttctctattttgctaccattttttttctggttaatattgcagataacctgattgttaaaaatgcctcctcagtcacttgagctccctctctttcatattcctgttttttttttttttttttaaaaacttctcctctctgtatctctaattctttactgattttctttctcggtacttttctcttgatatatctgtggcttctcccctcccttactttataccacatgggattaagtgcggagagcaggaagctgctttaaagagagtctcaaggggaatttcatcagccgcaagatgacatcacaagacagaaggaagtctccagccagcatcttcaaagtgcttgacaggctatctctcgtacctgcataagtggtgaatttccccctcagagtaaaccaggcaagaactagacagggcccctcccccgctgttagtaccttcccaaagctcaattagtcttacatcttctgtgcatggctcttgggagctggttagctttagccagatttcccttatggacaagagaaaaatggaacaaattccttgaatataagtatcatttacaaatagaggtaaggatttggcccccagaatttttgcaggatcagttttattaggtgattaaagcaaggcagatgatggaacaacaaagagaatcaccactgtaaaagccccccagtcctttgcccactaggtctgctttcctataatatgtctaggaggtgatgatattatcaaattcacagaatataatatgttctgcattcacttgtctgtcttaacacacacacacacacacacacacacacacacacacacacaccccaaatatttcttaaatagtcacctgttatttcccaagaacatcccaacttttccggtaggcaagtgtcaagtcacctggacaatattcctgaaaaaaagtgaaggttctacctggggttggggctcattttggaacatgaagttggcactctgaggagcctaaccttctgttaggcttcctttggggcagttggtttgcagtgatccttaataaaatagtttgcagtattttgtaggaaggatggctgtgtactcatgtgcagggaaaattaaactgctgcaagagtttggcaaaaccgccaccactttactattaatatgtctttactatttttatatgtgttttgtgggtcagcgtgttcaattttaaataggaatacactagccttacaacggaatttcttcttgtggaataaaaactgataataacctttcatcttcaagaatctgatgactgagttttgtaggaatgtacccaacagttggaccagagcctggccagttcccaccaattggagtaagctatttaattgagggttaagaagctcgatagttttttcaatctgttatcagaaaaatattatctaaagacactgatagcagctgactggtaaaactgaaagttggccttagtaaatggaactagtaaaaaatagataaaaatatgtaaaaccttttatgtcctgtcaaggaaaaaaaaataagcaaacttgatttgttttaaaattgtaaatattcattgcaaaacctaaattatacaaaatctatttcgatttgaatctcttaacatatgaaaagccacatcttaagtgcattatgctctgtaaggcatgttattagccagctttgctgaaaaatgtattcttctacagctcagaaagaggagtaaaattgctctagaaatttttgatctcagccactttgcatccaaagaggaacccaactccccactcacaatcccccacacacattttaattataagagttcaacattttccacagatatcattaagccaagcaaggaaactcctctctgtaaaaacaacaaaaaagttgcaatgtaaatagttattttagctattttctttgtgattagaggataagattttgttatttcttttggtacttagtcctttaactatctgaaaatggctcactttgacctgtttctttggtcagccaaactgaagtgaggggcctctgtttatatatagaatcttgtatatgtaactttacttatgtgggaccaatggccagtatattctaatggacacaaatctcccaatccttatgtaggattatctgttcaccttgccatgtccatcttgcaatgagacctaacttgcacctgcctgtttttctgcatgattggactccaaagactattaccaatgatgagaattgtcttgtgtcagtgtttataaaatggacttaaagcacacacacatgcatacccacattcacatgcacccttacagttctagatcaaccctggaaggcaaaaccatgtacgtccagtttctttgcaaggtctgagcagtgagaactaaattcagtcagtctaatgggtaaaatgcaacctcagtgatgcttaataaggatttgaaatcgttcaagtcatttctgtgttctttctcacaccctttcctttctattttgtcttcttttattttcaaaatgtctgtggaacaagaggatgaatcgggggagaacttctgcttcttttgactctttttaggtaatatggagtcaatatattatgatttctgtttcttatggattattaaaatcagaggaaatctgcttctctcaacataaaaccaaatccaaacccagacattgttggagtttgcctgttgacaatggttatgatgctgctcgttgtcttgagttgcatgtaacgacagtagctctgaagggtgtcatgatttcattttggcaactggtaaaagaaacttgtagtgtggtctgattgtgccagggaataaataagaaataaggaatgaaaaaaaaaaaaaaaaaaa

SEQ ID NO: 5 - DNA, 6466 bp, Homo sapiens
gctagtgtgagaaacaccaacagcaggcccatctcagatcttcactatggcaacttatgcaagaaactgttgaattagacccgtttcctatagatgagaaaccatacaagctgtggtatttatgagcctccatttcttatactactgcagtgaaccaacattggatgtgaaaattgccttttgtcagggattcgataaacaagtggatgtgtcatatattgccaaacattacaacatgagcaaaagcaaagttgacaaccagttctacagtgtggaagtgggagactcaaccttcacagttctcaagcgctaccagaatctaaagcctattggctctggggctcagggcatagtttgtgccgcgtatgatgctgtccttgacagaaatgtggccattaagaagctcagcagaccctttcagaaccaaacacatgccaagagagcgtaccgggagctggtcctcatgaagtgtgtgaaccataaaaacattattagtttattaaatgtcttcacaccccagaaaacgctggaggagttccaagatgtttacttagtaatggaactgatggatgccaacttatgtcaagtgattcagatggaattagaccatgagcgaatgtcttacctgctgtaccaaatgttgtgtggcattaagcacctccattctgctggaattattcacagggatttaaaaccaagtaacattgtagtcaagtctgattgcacattgaaaatcctggactttggactggccaggacagcaggcacaagcttcatgatgactccatatgtggtgacacgttattacagagcccctgaggtcatcctggggatgggctacaaggagaacgttgacatgtggtcagtagggtgcatcatgggagaaatgataaaaggtgcagtgctgtttcctggcactgatcatattgaccagtggaataaggtaattgaacaactaggaacaccatgtccagaattcatgaagaaattgcaacccacagtaagaaactatgtggagaatcggcccaagtatgcgggactcaccttccccaaactcttcccagattccctcttcccagcggactccgagcacaataaactcaaagccagccaagccagggacttgttgtcaaagatgctagtgattgacccagcaaaaagaatatcagtggacgacgccttacagcatccctacatcaacgtctggtatgacccagccgaagtggaggcgcctccacctcagatatatgacaagcagttggatgaaagagaacacacaattgaagaatggaaagaacttatctacaaggaagtaatgaattcagaagaaaagactaaaaatggtgtagtaaaaggacagccttctccttcaggtgcagcagtgaacagcagtgagagtctccctccatcctcgtctgtcaatgacatctcctccatgtccaccgaccagaccctggcatctgacactgacagcagcctggaagcctcggcaggacccctgggttgttgcaggtgactagccgcctgcctgcgaaacccagcgttcttcaggagatgatgtgatggaacacacacacacgcagacacacacacacacacaaatgcagacacacaacatcaagaaaacagcaagggagagaatccaagcctaaaattaaataaatctttcagcctgcttcttccccagggttctgtattgcagctaagctcaaatgtatatttaacttctagttgctcttgctttggtcttcttccaatgatgcttactacagaaagcaaatcagacacaattagagaagccttttccataaagtgtaattttaatggctgcaaaaccggcaacctgtaactgcccttttaaatggcatgacaaggtgtgcagtggccccatccagcatgtgtgtgtctctatcttgcatctacctgctccttggcctagtcagatggatgtagatacagatccgcatgtgtctgtattcatacagcactacttacttagagatgctactgtcagtgtcctcagggctctaccaagacataatgcactggggtaccacatggtccatttcatgtgatctattactctgacataaacccatctgtaatatattgccagtatataagctgtttagtttgttaattgattaaactgtatgtcttataagaaaacatgtaaagggggaatatatggggggagtgagctctctcagacccttgaagatgtagcttccaaatttgaatggattaaatggcacctgtataccaatttgtagaaagaacatatgtgatacttatgtaaagtgtggggcgagtgggtaacagttttcaggcacaaaatggtttggccttctgaaggcaggtgtgaataaaagctgagagtcttttctgtgacagtaaagagtaggagagactggagggtagtgtgtggtgtggactaagactgaaaggaggttattacccagatactaggagacactggtgtccctgttttagagatttggctctatacccatttgtcatttctcaattactggtcacaacaagaggccactggagaaagtggcaggtggtgaagcaaggttattgtgtgtgcttttgatgataagaatctctctctggaactgagcagaattgcctccatgtgaattccttcaacaaaaaaatgagcctgcctagactactttcagattgtggattgtgttttccatgtgtgggctgcttccctgattcatactctgccaaagttttattttgagaaataatattactttcctcttctgaaataaaataataataataataatgaaacccccaaaccacagtgtgagtctcaggttagcatttgaaaacatctccagagacattgttattcctcaggagtttccctgactccttaatgtggctgatgtttcatgtttatttatttattttaataagtatgagcaatcgaagggctgatcatctgagttttgtactgcagtagatgtaagagcaataactctgcccataaatttactgctgccaaatagttttagcaatatgagtttgtcttaaaagccatgaagcattaatgtcaatgtttagcaaaatctccagaatttagcttaaattaaattaaatgaaagtgctttcctgagcacttaaggaggcactgtgaaacatcttggttatgtacaaggcaataccaaggtgatctcagcaataatcaggcttcccttcttattcacttcgaagtatccatcacagttatttgcactctgaccatgttggtgctcactagagtggtgatatttcacctgaagaagtgaaagacaggaagtggatggttgatgagtgtgaactgattggcaaatagaatatccccaaaacagagcaaacaagaatgccagtgctctgaaaactgccttaaatataataatagggaactttctttctctcttcaagcaatagagaccacagggaactgcacccacctgcaccatcgccatgccagtggtgctgccattctgagattatttcacacagtcagattcagtacacgcagtcatttgtttaaaataaatagaagaacattcctttgtgcagcttgtcccctggaataaaaatggcccattttctctattttgctaccattttttttctggttaatattgcagataacctgattgttaaaaatgcctcctcagtcacttgagctccctctctttcatattcctgttttttttttttttttttaaaaacttctcctctctgtatctctaattctttactgattttctttctcggtacttttctcttgatatatctgtggcttctcccctcccttactttataccacatgggattaagtgcggagagcaggaagctgctttaaagagagtctcaaggggaatttcatcagccgcaagatgacatcacaagacagaaggaagtctccagccagcatcttcaaagtgcttgacaggctatctctcgtacctgcataagtggtgaatttccccctcagagtaaaccaggcaagaactagacagggcccctcccccgctgttagtaccttcccaaagctcaattagtcttacatcttctgtgcatggctcttgggagctggttagctttagccagatttcccttatggacaagagaaaaatggaacaaattccttgaatataagtatcatttacaaatagaggtaaggatttggcccccagaatttttgcaggatcagttttattaggtgattaaagcaaggcagatgatggaacaacaaagagaatcaccactgtaaaagccccccagtcctttgcccactaggtctgctttcctataatatgtctaggaggtgatgatattatcaaattcacagaatataatatgttctgcattcacttgtctgtcttaacacacacacacacacacacacacacacacacacacacacaccccaaatatttcttaaatagtcacctgttatttcccaagaacatcccaacttttccggtaggcaagtgtcaagtcacctggacaatattcctgaaaaaaagtgaaggttctacctggggttggggctcattttggaacatgaagttggcactctgaggagcctaaccttctgttaggcttcctttggggcagttggtttgcagtgatccttaataaaatagtttgcagtattttgtaggaaggatggctgtgtactcatgtgcagggaaaattaaactgctgcaagagtttggcaaaaccgccaccactttactattaatatgtctttactatttttatatgtgttttgtgggtcagcgtgttcaattttaaataggaatacactagccttacaacggaatttcttcttgtggaataaaaactgataataacctttcatcttcaagaatctgatgactgagttttgtaggaatgtacccaacagttggaccagagcctggccagttcccaccaattggagtaagctatttaattgagggttaagaagctcgatagttttttcaatctgttatcagaaaaatattatctaaagacactgatagcagctgactggtaaaactgaaagttggccttagtaaatggaactagtaaaaaatagataaaaatatgtaaaaccttttatgtcctgtcaaggaaaaaaaaataagcaaacttgatttgttttaaaattgtaaatattcattgcaaaacctaaattatacaaaatctatttcgatttgaatctcttaacatatgaaaagccacatcttaagtgcattatgctctgtaaggcatgttattagccagctttgctgaaaaatgtattcttctacagctcagaaagaggagtaaaattgctctagaaatttttgatctcagccactttgcatccaaagaggaacccaactccccactcacaatcccccacacacattttaattataagagttcaacattttccacagatatcattaagccaagcaaggaaactcctctctgtaaaaacaacaaaaaagttgcaatgtaaatagttattttagctattttctttgtgattagaggataagattttgttatttcttttggtacttagtcctttaactatctgaaaatggctcactttgacctgtttctttggtcagccaaactgaagtgaggggcctctgtttatatatagaatcttgtatatgtaactttacttatgtgggaccaatggccagtatattctaatggacacaaatctcccaatccttatgtaggattatctgttcaccttgccatgtccatcttgcaatgagacctaacttgcacctgcctgtttttctgcatgattggactccaaagactattaccaatgatgagaattgtcttgtgtcagtgtttataaaatggacttaaagcacacacacatgcatacccacattcacatgcacccttacagttctagatcaaccctggaaggcaaaaccatgtacgtccagtttctttgcaaggtctgagcagtgagaactaaattcagtcagtctaatgggtaaaatgcaacctcagtgatgcttaataaggatttgaaatcgttcaagtcatttctgtgttctttctcacaccctttcctttctattttgtcttcttttattttcaaaatgtctgtggaacaagaggatgaatcgggggagaacttctgcttcttttgactctttttaggtaatatggagtcaatatattatgatttctgtttcttatggattattaaaatcagaggaaatctgcttctctcaacataaaaccaaatccaaacccagacattgttggagtttgcctgttgacaatggttatgatgctgctcgttgtcttgagttgcatgtaacgacagtagctctgaagggtgtcatgatttcattttggcaactggtaaaagaaacttgtagtgtggtctgattgtgccagggaataaataagaaataaggaatgaaaaaaaaaaaaaaaaaaa

SEQ ID NO: 6 - DNA, 6561 bp, Homo sapiens
aaccataattatcatgtgatctagacctgatacattgtagatgaagaaacatgtcgacagagccactatctgcccaacatctgatgggtagtcatagtcacagtggaactgagactaaggtttccatttttatttgactatgcttcttcccaaggctagtgtgagaaacaccaacagcaggcccatctcagatcttcactatggcaacttatgcaagaaactgttgaattagacccgtttcctatagatgagaaaccatacaagctgtggtatttatgagcctccatttcttatactactgcagtgaaccaacattggatgtgaaaattgccttttgtcagggattcgataaacaagtggatgtgtcatatattgccaaacattacaacatgagcaaaagcaaagttgacaaccagttctacagtgtggaagtgggagactcaaccttcacagttctcaagcgctaccagaatctaaagcctattggctctggggctcagggcatagtttgtgccgcgtatgatgctgtccttgacagaaatgtggccattaagaagctcagcagaccctttcagaaccaaacacatgccaagagagcgtaccgggagctggtcctcatgaagtgtgtgaaccataaaaacttacttagtaatggaactgatggatgccaacttatgtcaagtgattcagatggaattagaccatgagcgaatgtcttacctgctgtaccaaatgttgtgtggcattaagcacctccattctgctggaattattcacagggatttaaaaccaagtaacattgtagtcaagtctgattgcacattgaaaatcctggactttggactggccaggacagcaggcacaagcttcatgatgactccatatgtggtgacacgttattacagagcccctgaggtcatcctggggatgggctacaaggagaacgttgacatgtggtcagtagggtgcatcatgggagaaatgataaaaggtgcagtgctgtttcctggcactgatcatattgaccagtggaataaggtaattgaacaactaggaacaccatgtccagaattcatgaagaaattgcaacccacagtaagaaactatgtggagaatcggcccaagtatgcgggactcaccttccccaaactcttcccagattccctcttcccagcggactccgagcacaataaactcaaagccagccaagccagggacttgttgtcaaagatgctagtgattgacccagcaaaaagaatatcagtggacgacgccttacagcatccctacatcaacgtctggtatgacccagccgaagtggaggcgcctccacctcagatatatgacaagcagttggatgaaagagaacacacaattgaagaatggaaagaacttatctacaaggaagtaatgaattcagaagaaaagactaaaaatggtgtagtaaaaggacagccttctccttcaggtgcagcagtgaacagcagtgagagtctccctccatcctcgtctgtcaatgacatctcctccatgtccaccgaccagaccctggcatctgacactgacagcagcctggaagcctcggcaggacccctgggttgttgcaggtgactagccgcctgcctgcgaaacccagcgttcttcaggagatgatgtgatggaacacacacacacgcagacacacacacacacacaaatgcagacacacaacatcaagaaaacagcaagggagagaatccaagcctaaaattaaataaatctttcagcctgcttcttccccagggttctgtattgcagctaagctcaaatgtatatttaacttctagttgctcttgctttggtcttcttccaatgatgcttactacagaaagcaaatcagacacaattagagaagccttttccataaagtgtaattttaatggctgcaaaaccggcaacctgtaactgcccttttaaatggcatgacaaggtgtgcagtggccccatccagcatgtgtgtgtctctatcttgcatctacctgctccttggcctagtcagatggatgtagatacagatccgcatgtgtctgtattcatacagcactacttacttagagatgctactgtcagtgtcctcagggctctaccaagacataatgcactggggtaccacatggtccatttcatgtgatctattactctgacataaacccatctgtaatatattgccagtatataagctgtttagtttgttaattgattaaactgtatgtcttataagaaaacatgtaaagggggaatatatggggggagtgagctctctcagacccttgaagatgtagcttccaaatttgaatggattaaatggcacctgtataccaatttgtagaaagaacatatgtgatacttatgtaaagtgtggggcgagtgggtaacagttttcaggcacaaaatggtttggccttctgaaggcaggtgtgaataaaagctgagagtcttttctgtgacagtaaagagtaggagagactggagggtagtgtgtggtgtggactaagactgaaaggaggttattacccagatactaggagacactggtgtccctgttttagagatttggctctatacccatttgtcatttctcaattactggtcacaacaagaggccactggagaaagtggcaggtggtgaagcaaggttattgtgtgtgcttttgatgataagaatctctctctggaactgagcagaattgcctccatgtgaattccttcaacaaaaaaatgagcctgcctagactactttcagattgtggattgtgttttccatgtgtgggctgcttccctgattcatactctgccaaagttttattttgagaaataatattactttcctcttctgaaataaaataataataataataatgaaacccccaaaccacagtgtgagtctcaggttagcatttgaaaacatctccagagacattgttattcctcaggagtttccctgactccttaatgtggctgatgtttcatgtttatttatttattttaataagtatgagcaatcgaagggctgatcatctgagttttgtactgcagtagatgtaagagcaataactctgcccataaatttactgctgccaaatagttttagcaatatgagtttgtcttaaaagccatgaagcattaatgtcaatgtttagcaaaatctccagaatttagcttaaattaaattaaatgaaagtgctttcctgagcacttaaggaggcactgtgaaacatcttggttatgtacaaggcaataccaaggtgatctcagcaataatcaggcttcccttcttattcacttcgaagtatccatcacagttatttgcactctgaccatgttggtgctcactagagtggtgatatttcacctgaagaagtgaaagacaggaagtggatggttgatgagtgtgaactgattggcaaatagaatatccccaaaacagagcaaacaagaatgccagtgctctgaaaactgccttaaatataataatagggaactttctttctctcttcaagcaatagagaccacagggaactgcacccacctgcaccatcgccatgccagtggtgctgccattctgagattatttcacacagtcagattcagtacacgcagtcatttgtttaaaataaatagaagaacattcctttgtgcagcttgtcccctggaataaaaatggcccattttctctattttgctaccattttttttctggttaatattgcagataacctgattgttaaaaatgcctcctcagtcacttgagctccctctctttcatattcctgttttttttttttttttttaaaaacttctcctctctgtatctctaattctttactgattttctttctcggtacttttctcttgatatatctgtggcttctcccctcccttactttataccacatgggattaagtgcggagagcaggaagctgctttaaagagagtctcaaggggaatttcatcagccgcaagatgacatcacaagacagaaggaagtctccagccagcatcttcaaagtgcttgacaggctatctctcgtacctgcataagtggtgaatttccccctcagagtaaaccaggcaagaactagacagggcccctcccccgctgttagtaccttcccaaagctcaattagtcttacatcttctgtgcatggctcttgggagctggttagctttagccagatttcccttatggacaagagaaaaatggaacaaattccttgaatataagtatcatttacaaatagaggtaaggatttggcccccagaatttttgcaggatcagttttattaggtgattaaagcaaggcagatgatggaacaacaaagagaatcaccactgtaaaagccccccagtcctttgcccactaggtctgctttcctataatatgtctaggaggtgatgatattatcaaattcacagaatataatatgttctgcattcacttgtctgtcttaacacacacacacacacacacacacacacacacacacacacaccccaaatatttcttaaatagtcacctgttatttcccaagaacatcccaacttttccggtaggcaagtgtcaagtcacctggacaatattcctgaaaaaaagtgaaggttctacctggggttggggctcattttggaacatgaagttggcactctgaggagcctaaccttctgttaggcttcctttggggcagttggtttgcagtgatccttaataaaatagtttgcagtattttgtaggaaggatggctgtgtactcatgtgcagggaaaattaaactgctgcaagagtttggcaaaaccgccaccactttactattaatatgtctttactatttttatatgtgttttgtgggtcagcgtgttcaattttaaataggaatacactagccttacaacggaatttcttcttgtggaataaaaactgataataacctttcatcttcaagaatctgatgactgagttttgtaggaatgtacccaacagttggaccagagcctggccagttcccaccaattggagtaagctatttaattgagggttaagaagctcgatagttttttcaatctgttatcagaaaaatattatctaaagacactgatagcagctgactggtaaaactgaaagttggccttagtaaatggaactagtaaaaaatagataaaaatatgtaaaaccttttatgtcctgtcaaggaaaaaaaaataagcaaacttgatttgttttaaaattgtaaatattcattgcaaaacctaaattatacaaaatctatttcgatttgaatctcttaacatatgaaaagccacatcttaagtgcattatgctctgtaaggcatgttattagccagctttgctgaaaaatgtattcttctacagctcagaaagaggagtaaaattgctctagaaatttttgatctcagccactttgcatccaaagaggaacccaactccccactcacaatcccccacacacattttaattataagagttcaacattttccacagatatcattaagccaagcaaggaaactcctctctgtaaaaacaacaaaaaagttgcaatgtaaatagttattttagctattttctttgtgattagaggataagattttgttatttcttttggtacttagtcctttaactatctgaaaatggctcactttgacctgtttctttggtcagccaaactgaagtgaggggcctctgtttatatatagaatcttgtatatgtaactttacttatgtgggaccaatggccagtatattctaatggacacaaatctcccaatccttatgtaggattatctgttcaccttgccatgtccatcttgcaatgagacctaacttgcacctgcctgtttttctgcatgattggactccaaagactattaccaatgatgagaattgtcttgtgtcagtgtttataaaatggacttaaagcacacacacatgcatacccacattcacatgcacccttacagttctagatcaaccctggaaggcaaaaccatgtacgtccagtttctttgcaaggtctgagcagtgagaactaaattcagtcagtctaatgggtaaaatgcaacctcagtgatgcttaataaggatttgaaatcgttcaagtcatttctgtgttctttctcacaccctttcctttctattttgtcttcttttattttcaaaatgtctgtggaacaagaggatgaatcgggggagaacttctgcttcttttgactctttttaggtaatatggagtcaatatattatgatttctgtttcttatggattattaaaatcagaggaaatctgcttctctcaacataaaaccaaatccaaacccagacattgttggagtttgcctgttgacaatggttatgatgctgctcgttgtcttgagttgcatgtaacgacagtagctctgaagggtgtcatgatttcattttggcaactggtaaaagaaacttgtagtgtggtctgattgtgccagggaataaataagaaataaggaatgaaaaaaaaaaaaaaaaaaa

(19) United States
(12) Patent Application Publication
(10) Pub. No.: US 2019/0381125 A1
(43) Pub. Date: Dec. 19, 2019

(54) Methods of Treating Angiogenesis-Related Disorders Using JNK3 Inhibitors

(71) Applicant: University of Massachusetts, Boston, MA (US)

(72) Inventors: Roger J. Davis, Princeton, MA (US); John F. Keaney, JR., Hingham, MA (US); Kasmir Ramo, Worcester, MA (US)

(21) Appl. No.: 16/485,704

(22) PCT Filed: Feb. 14, 2018
(86) PCT No.: PCT/US2018/018209

§ 371 Date: Aug. 13, 2019

Related U.S. Application Data
(60) Provisional application No. 62/459,381, filed on Feb. 15, 2017.

(51) Int. Cl. A61K 38/00; C12N 15/113; A61P 9/10

(52) U.S. Cl. CPC A61K 38/005; C12N 15/1137; C12N 2310/14; C12Y 207/11024; A61P 9/10

(57) ABSTRACT

The disclosure provides methods and compositions for treating angiogenesis-related disorders, e.g., peripheral arterial disease (PAD), tissue ischemia, etc., using an agent that inhibits C-Jun N-terminal kinase 3 (JNK3) expression and/or activity. Specifically, the disclosure provides methods of treating angiogenesis-related disorders in a subject, the methods comprising administering to the subject a therapeutically effective amount of an agent that inhibits JNK3 expression and/or activity, wherein the agent is an inhibitory nucleic acid, a peptide, or peptide-inhibitor.

### CLAIM OF PRIORITY

This application claims the benefit of U.S. Provisional Patent Application Ser. No. 62/459,381, filed on Feb. 15, 2017. The entire contents of the foregoing are hereby incorporated by reference.

### FEDERALLY SPONSORED RESEARCH OR DEVELOPMENT

This invention was made with Government support under Grant Nos. HL092122, HL098407 and DK107220 awarded by the National Institutes of Health. The Government has certain rights in the invention.

### TECHNICAL FIELD

Described herein are methods and compositions comprising JNK3 inhibitors, and methods of use thereof, e.g., to treat angiogenesis-related disorders (e.g., peripheral arterial disease (PAD), tissue ischemia, etc.).

### BACKGROUND

Angiogenesis is an essential physiological process that leads to new blood vessel is formation during embryogenesis, tissue growth and repair, and tumorigenesis (Folkman Nat Med 1995; 1:27-31). Abnormally enhanced angiogenesis is implicated in many diseases such as cancer, which could be a potential target for therapy. Thus far, a number of anti-angiogenic agents have been approved by FDA with focus mainly on inhibiting the vascular endothelial growth factor (VEGF) pathway and more are now in clinical development. On the contrary, diseases such as ischemic heart or limbs could benefit from a promoted angiogenesis. Numerous angiogenic growth factors have been discovered as potential therapeutic agents, however studies have not yet led to clinical approval (Kaminsky S M, et al., Human Gene Therapy 2013; 24: 948).

### SUMMARY

Peripheral arterial disease (PAD) refers to a diverse group of disorders that produces progressive stenosis, occlusion or aneurysmal dilatation of non-coronary vessels (Hirsch et al., J Am Coil Cardiol. 2006; 47:1239-1312). PAD is often due to atherosclerosis and its manifestation in the extremities commonly results in limb ischemia and pain (claudication) with exercise, largely due to inadequate formation of collateral vessels. In advanced stages, pain is manifest at rest producing critical limb ischemia that threatens limb viability. This condition impacts up to 14% of patients aged 70 and older and is particularly prevalent in those with type 2 diabetes mellitus (Hirsch et al., J Am Coll Cardiol. 2006; 47:1239-1312). In this latter population, impaired wound healing is also a significant clinical problem resulting from vascular insufficiency. Since the prevalence of diabetes and patients aged >70 is growing, PAD is destined to become an increasing public health problem (Hirsch et al., J Am Coll Cardiol. 2006; 47:1239-1312). More than 200,000 individuals undergo lower-limb amputation every year because of peripheral vascular diseases (Hirsch et al., J Am Coll Cardiol. 2006; 47:1239-1312). Clinical manifestations of PAD (claudication and limb ischemia) have limited medical treatment options, with only two currently approved medications (cilostazol and pentoxifylline) that have modest impact on the disease. Thus, there is a great unmet need for treatment of PAD, and a clear need to understand the molecular mechanisms which lead to increased blood flow in these patients.

Tissue ischemia (hypoxia), such as that observed with PAD, elicits a number of adaptive responses. In very early stages, metabolic autoregulation facilitates local vasodilation in an attempt to maximize tissue perfusion and mitigate acute tissue hypo-perfusion. Soon thereafter, genetic programs under the control of hypoxia inducible factor-1α (HIF-1α)(Semenza, Cell. 2012; 148:399-408) and peroxisome proliferator gamma coactivator-1α (PGC-1α) (Arany et al., Nature. 2008; 451:1008-1012) are activated and lead to tissue stress resistance and the recruitment of additional vascular capacity via vasodilation and new vessel formation as a means to durably enhance tissue perfusion and viability. Key genes upregulated in this manner include vascular endothelial growth factor-A (VEGF-A), its receptors (VEGFR1-2), angiopoietin-like 4 protein (Angpt1-4), and estrogen related receptor alpha (ERR-α) (Arany et al., Nature. 2008; 451:1008-1012; Semenza Cell. 2012; 148:399-408). These genes coordinate activation of endothelial cells to sprout, form tip cells, and migrate towards the hypoxic tissue as initial steps in forming new vessels (Fraisl et al., Dev Cell. 2009; 16:167-179). Thus, stress-responsive pathways are important in coordinating the tissue response to ischemia.

C-Jun N-terminal kinases (JNKs) are members of mitogen-activated protein kinase family and involve stress responses. Three different JNK genes (jnk1, jnk2 and jnk3) encoding 10 splice variants have been identified, leading to four forms each for JNK1 and JNK2, and 2 JNK3 gene products (Gupta et al., EMBO J. 1996; 15:2760-2770). JNK1 and JNK2 isoforms are widely expressed in organs/tissues, whereas JNK3 is predominantly expressed in the central nervous system (Manning & Davis, Nat Rev Drug Discov 2003; 2: 554-565) and, to a much less extent, in the heart and testes. The role of JNKs is not well understood and continuously evolving. It is known that JNKs act in the apoptosis cascade interacting with multiple pro- and anti-apoptotic proteins as well as metabolic syndrome through endoplasmic reticulum stress (Weston C R and Davis R J. Curr Opin Cell Bio 2007; 19: 142-9). Mice lacking JNK1 have shown decreased adiposity and increased insulin sensitivity (Hirosumi J, et al., Nature 2002; 420: 333-6). JNK2 and JNK3 and dual JNK2/3 knockout mice were resistant to motor neuron degeneration in a mouse model of Parkinson's disease (Hunot, S et al., Proc. Natl. Acad. Sci. USA 2004; 101: 665-70). More interestingly, JNK3 null mice exhibited an attenuated neuronal death induced by β-amyloid, implicating a role of JNK3 in Alzheimer's disease (Morishima Y, et al., J Neurosci 2001; 21: 7551-60).

Provided herein are methods of treating angiogenesis-related disorders in a subject that include administering to the subject a therapeutically effective amount of an agent that inhibits JNK3 expression and/or activity.

In some embodiments, the agent is a small molecule or an inhibitory nucleic acid. In some embodiments, the inhibitory nucleic acid is an antisense molecule, a small interfering RNA, or a small hairpin RNA which are specific for a nucleic acid encoding SEQ ID NO: 1. In some embodiments, the inhibitory nucleic acid is a nucleic acid comprising a sequence that is complementary to a contiguous sequence, e.g., a contiguous sequence of at least 5 nucleotides present in JNK3.

In some embodiments, the agent is a peptide or peptide-inhibitor. In some embodiments, the peptide inhibitor is a peptide JNK3 inhibitor.

In some embodiments, the subject is human. In some embodiments, the subject has, or is at risk of having an angiogenesis-related disorder. In some embodiments, the subject has peripheral arterial disease (PAD), macular degeneration, retinopathy, stroke, diabetic limb ulcers, diabetic neuropathy, age-related blindness, chronic wounds, pressure ulcers, Alzheimer's disease, myocardial ischemia, cerebral ischemia, hepatic ischemia, limb ischemia, pulmonary ischemia, renal ischemia, testicular ischemia, intestinal type ischemia, or any organ ischemia.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIG. 1A: JNK1 and JNK2 mRNA expression in wild-type (WT) and JNK3^{-/-} mice.

FIG. 1B: JNK3 mRNA expression from femoral nerves harvested from control and ischemic legs.

FIG. 1C: JNK1, JNK2, and JNK3 mRNA expression from peripheral nerves harvested from control and ischemic legs.

FIG. 1D: Time course of blood flow recovery by Laser Doppler Imaging in HLI (n=6 in each group).

FIG. 1E: Representative figures of blood flow measurements by Laser Doppler Imaging in hind limb ischemia model for day 3 and Day 7 post HLI WT and JNK3^{-/-} mice.

FIG. 1F: Expression of CD31 in mouse gastrocnemius muscle on Day 21 after ligation of femoral artery.

FIG. 1G: Quantification of angiogenesis marker, CD31, in mouse gastrocnemius muscle on Day 21 after ligation of femoral artery. Statistically significant differences between groups are indicated (*, P<0.05).

FIG. 1H: Gastrocnemius muscle was harvested from ischemic or non-ischemic legs 3 days after hind limb ischemia in wild-type (WT) or JNK3^{-/-} mice. Sections were taken and stained for nerve integrity using S-100 antibody.

FIG. 1I: Time course of blood flow recovery by Laser Doppler Imaging in hind limb ischemia model. P<0.01 by two-way ANOVA; n=5-7 in each group.

FIG. 1J: Angiopoietin 1 (Angpt1), Angiopoietin 2 (Angpt2), Estrogen-related receptor alpha (ERRα) and Fetal liver kinase-1 (FLK1) mRNA expression in gastrocnemius muscle at day 7 vs day 0 after hind limb ischemia in wild-type (WT) and JNK3^{-/-} mice. P<0.05 by ANOVA with post-hoc Turkey test; n=5.

FIG. 1K: Blood flow recovery from hind limb ischemia by genotype; n=5 per group. SARM1=Sterile Alpha And TIR Motif Containing 1.

FIG. 1L: JNK activation (total pJNK1/2/3) with hypoxia as a function of the Jnk3 siRNA

FIG. 1M: Vascular endothelial growth factor A (Vegfa) mRNA expression as a function of hypoxia (1% O₂ 24 h) with or without suppression of Jnk3; * p<0.05 by ANOVA; n=4.

FIG. 2A: Suppression of JNK3 activates pro-angiogenic factors in gastrocnemius muscle during ischemia. Microarray was performed on pooled (3 mice) gastrocnemius muscle three days post femoral artery ligation.

FIGS. 2B-2E: real-time quantitative reverse transcription polymerase chain reaction (qRT-PCR) was performed for (2B) different angiogenesis related genes, (2C) transcription regulator genes, (2D) neuropeptide genes, (2E) mitochondrial biogenesis on gastrocnemius muscle from control and ischemic legs of WT and JNK3^{-/-} mice 3 days post femoral artery ligation. Statistically significant differences between groups are indicated (*, P<0.05).

FIGS. 2F-2G: Gastrocnemius muscle was isolated from control (Cont) or ischemic (Isch) hindlimb and immunoblot was performed with antibodies to (2F) Platelet-derived growth factor subunit B (PDGF-B) and (2G) VEGF-A.

FIG. 3A: JNK3 regulates pro-angiogenic factors via Creb1 transcription factor expression and activation. Femoral nerves were isolated on day 3 after femoral artery ligation and qRT-PCR was performed for different angiogenesis related genes.

FIG. 3B: Following one hour of hypoxia, RNA was isolated from control and JNK3 knockdown Neuro-2a (N2a) cells and qRT-PCR was performed for angiogenesis related genes.

FIG. 3C: qRT-PCR was performed for JNK3, Creb1, and PDGFb after siRNA delivery of Jnk3 into peripheral nerves. Statistically significant differences between groups are indicated (*, P<0.05).

FIG. 3D: JNK3 regulated Cyclic AMP-responsive element binding protein 1 (Creb1) but not Nuclear respiratory factor 1 (NRF1) controls Pdgfb and Heparin binding epidermal growth factor-like growth factor (Hbeg/) gene expression in neuro-2a cell line. RNAs were isolated from control and Jnk3 siRNA without or with Creb1 and Nrf1 siRNA and qRT-PCR was performed for genes as indicated.

FIG. 3E: Gastrocnemius muscle was isolated from control and ischemic legs of both WT and JNK3“mice on day 3 after femoral artery ligation and immunoblot analysis was performed with antibodies to pCreb1 (phospho-133), Creb1, and alpha-actin. Extracts prepared from WT and JNK3” brain were examined by immunoblot analysis with antibodies to Creb1 and actin.

FIG. 3F: Lysates prepared from N2a treated with control siRNA or siRNA against JNK3 (48 hours) were examined by immunoblot analysis using antibodies to Creb1 and actin.

FIG. 3G: A Luciferase reporter assay for the Creb1 recombinase enzyme was performed on N2a cells treated with either control siRNA or JNK3 siRNA (48 hours) both with and without Forskolin (30 min) a Creb1 activator. Statistically significant differences between groups are indicated (*, P<0.05).

FIG. 4A: JNK3 regulates the Sirtuin1/Creb1 axis to control hind limb blood flow recovery following ischemia. Control Ad-GFP and Ad-Creb1 were injected (single dose of 2×10⁸ c.f.u.) into gastrocnemius muscle of WT mice. Three days following the injection femoral artery ligation was performed and blood flow measurements by Laser Doppler Imaging (n=6-8 in each group) were obtained.

FIG. 4B: Representative images of hind limb blood flow of WT mice 3 and 7 days post femoral artery ligation and adenoviral (control Ad-GFP and Ad-Creb1) injection measured by Laser Doppler Imaging

FIG. 4C: Neuro-2a cells were treated with either control or JNK3 siRNA under normoxic or hypoxic conditions. Lysates were examined by immunoprecipitation with a control antibody (IgG) or with an antibody to Acetyl Lysine. The immunoprecipitates were examined by immunoblot analysis using antibodies to Creb1 and Actin.

FIG. 4D: Lysates prepared from the brains of control and JNK3^{-/-} mice were examined by immunoblot analysis using antibodies to phosphorylated Sirtulin 1 (pSirt1) and actin.

FIG. 4E: Lysates prepared from Neuro-2a treated with or without siRNA against sirtuin1 (48 hours) were examined by immunoblot analysis using antibodies to pCreb, Creb1, Sirt1 and actin.

FIG. 4F: Neuro-2a cells were treated with either control or Sirtulin1 siRNA. Lysates were examined by immunoprecipitation with a control antibody (IgG) or with an antibody to Acetyl Lysine. The immunoprecipitates were examined by immunoblot analysis using antibodies to Creb1 and Acetyl Lysine. The cell lysates were examined by probing with an antibody to actin.

FIG. 4G: A CRE Luciferase assay was performed for N2a cells treated with either control siRNA or Sirtulin1 siRNA (48 hours) in both the presence and absence of Forskolin a Creb1 activator (30 min). Statistically significant differences between groups are indicated (*, P<0.05).

FIG. 4H: Schematic of pathway from ischemia leading to angiogenesis and blood flow recovery. Ischemia promotes JNK3 activation, which activates SirT1. SirT1 inhibits Creb1, which in turn leads to the upregulation of Vegfα, Pdgfβ, Plgf and Hbegf. This upregulation leads to angiogenesis and blood flow recovery.

FIG. 5A: Jnk3 expression in human aortic endothelial cells (HAEC), human microvascular endothelial cells (HMVEC) and human umbilical vein endothelial cells (HUVEC); n=3.

FIG. 5B: Jnk3 expression in mouse lung endothelial cells (MLEC), mouse skeletal muscle microvascular endothelial cells (MSMMEC) and mouse brain cells as a positive control; n=3.

FIG. 5C: Jnk3 expression in bone marrow (BM) and blood; n=3.

FIG. 5D: Capillary sprouting assay in wild type (WT) and Jnk3^{-/-} aortic segments. Black and white (B&W) image bar=1000 μm, bright field/immunofluorescence bar=100 μm.

FIG. 5E: Composite data on isolectin B4 (iB4) positive sprout density; n=3 mice per genotype.

FIG. 6A: Jnk3 mRNA expression in muscle tissue of Jnk3^{fl/fl} mice, HSA-Jnk3^{fl/fl} mice with brain tissue as a positive control; n=3.

FIG. 6B: Ischemic blood flow recovery in skeletal muscle-specific Jnk3 gene excision (M^{Jk3-/-}) with control; n=5-7.

FIG. 6C: Ischemic blood flow recovery in nervous tissue-specific Jnk3 gene excision (N^{Jnk-/-}) with control; n=5-7, p<0.05 vs N^{Ctrk} by two-way ANOVA on ranks.

FIG. 6D: JNK3 protein in nervous tissue of WT, Jnk3^{-/-}, Jnk3^{fl/fl}, Jnk3^{HSA-Cre}, Jnk3^{fl/fl}, Jnk3^{NES-Cre}.

FIG. 6E: Sciatic nerve Jnk expression in proximal perfused (Ctrl) and distal ischemic (ischemia) regions in the hind limb ischemia model; * p<0.05 by ANOVA.

FIG. 6F: Sciatic nerve protein expression of JNK3, PDGFb and VEGFA in proximal perfused (P) vs. distal (D) ischemic areas from human leg.

FIG. 6G: VEGFa expression from samples used in FIG. 6F; n=2.

FIG. 7: Schematic of central paradigm. Ischemia promotes stress responses in peripheral neural tissue, upregulating angiogenesis responses (VEGF, Fkl1, Angpt, others). JNK3 activation limits nerve and glial adaptive responses and its loss enhances angiogenesis and tissue recovery.

FIG. 8: Images of wound closure in WT and Jnk3^{-/-} mice at day 0, day 2 and day 6. Granulation tissue is complexly in place by day 2 and closed by day 6 in Jnk3^{-/-} mice. Bar=2 mm.

FIG. 9: Egr1 mRNA expression in N2a cells with Jnk3 loss-of-function under normoxic and hypoxic conditions. n=4; P<0.05 by ANOVA.

### DETAILED DESCRIPTION

Peripheral arterial disease (PAD) affects nearly 10 million people in the United States alone (Go et al., Circulation 2013; 127: 143-152; Go et al., Circulation 2013; 127: e6-e245), yet patients with clinical manifestations of PAD (e.g. claudication and limb ischemia) have limited and ineffective treatment options (Hamburg & Balady, Circulation 2011; 123: 87-97). In ischemic tissues, stress kinases, c-Jun N-terminal kinase (JNK), are activated. Without wishing to be bound by theory, the present results provide evidence that inhibition of the JNK3 isoform which is most highly expressed in the peripheral nerves, strikingly potentiates angiogenesis and blood flow recovery from mouse hind limb ischemia. JNK3 deficiency leads to increased pro-angiogenetic growth factors such as Vegfα, Pdgfb, Plgf Hbegfand Tgfb3 in ischemic muscle and cells by repression of the transcription factor Creb1. JNK3 acts through sirtuin 1 (SirT1) to suppress the activity of Creb1. With SirT1 suppression, as in JNK3-deficient mice, Creb1 is more active and upregulates pro-angiogenic factors. Together these data suggest that the JNK3/Sirtulin1/Creb1 axis coordinate the vascular remodeling response in peripheral ischemia.

Among the stress-induced pathways activated during ischemia, those relevant to angiogenesis are the c-Jun N-terminal kinase (JNK) family of protein kinases (Guma et al., Proc Natl Acad Sci USA 2009; 106: 8760-8765; Du et al., Proc Natl Acad Sci USA 2013; 110: 2377-2382). Three separate genes encode JNK1, JNK2, and JNK3, and alternative splicing can produce 10 different protein sequences with large homology (Davis, Cell 2000; 103: 239-252; Coffey, Nat Rev Neurosci 2014; 15: 285-299). JNK1 and JNK2 are expressed ubiquitously but JNK3 expression is mainly confined to neurons, the testis and the heart (Davis, Cell 2000; 103: 239-252; Coffey, Nat Rev Neurosci 2014; 15: 285-299). Alterations in JNK1 and JNK2 signaling are now well-established causes of metabolic diseases such as diabetes and chronic inflammation (Das et al., Cell 2009; 136: 249-260; Sabio & Davis, Trends Biochem Sci 2010; 35: 490-496; Han et al., Science 2013, 339(6116): 218-222), which have been further linked to the onset and progression of cardiovascular disease. A recent study implicated JNK1 and JNK2 in angiogenesis during development (Ramo et al., Elife 2016; 5), but the role of JNK3 in the regulation of neovascularization is not known.

### JNK3

The c-Jun N-terminal kinase (JNK) family of protein kinases are among the stress pathways activated in ischemic tissue and may be relevant to angiogenesis (Guma et al., Proc Natl Acad Sci USA 2009; 106: 8760-8765; Du et al., Proc Natl Acad Sci USA 2013; 110: 2377-2382). These kinases are derived from three gene products (JNK1, JNK2, JNK3) known to be activated in the setting of cellular injury (Kuan et al., Proc Natl Acad Sci USA. 2003; 100:15184-15189; Derijard et al., Cell. 1994; 76:1025-1037; Gupta et al., EMBO J. 1996; 15:2760-2770). The function of JNK isoforms is contextual, but they typically exert their influence via phosphorylation of transcription factors (e.g. c-Jun, AP-1, ATF2, etc.) and JNKs have been implicated in inflammation, apoptosis, and tissue repair after injury (Manning & Davis, Nat Rev Drug Discov 2003; 2: 554-565).

As described herein, JNK3 (also known as c-Jun N-terminal Kinase 3, stress activated protein kinase beta, or mitogen-activated protein kinase 10 (MAPK10)) is a member of mitogen-activated protein kinase family. JNK3 promotes stress-induced apoptotic neuronal death by several mechanisms (Manning & Davis, Nat Rev Drug Discov 2003; 2: 554-565) including inactivation of apoptosis inhibitors (Bcl-xL and Bcl-2) (Kharbanda et al., J Biol Chem. 2000; 275:322-327; Gross et al., Genes Dev. 1999; 13:1899-1911) and upregulation of Fas-ligand (Morishima et al., J Neurosci. 2001; 21:7551-7560) among others. This is the first demonstration of an anti-angiogenic property of JNK3.

Inhibitory nucleic acids useful in the present methods and compositions include those that are designed to target JNK3. Exemplary sequences of human JNK3 are provided in Table 1.

TABLE 1
Human JNK3 Sequences

| GeneBank | | |
|---|---|---|
| Accession Number | Name | SEQ ID NO |
| NM_138982.3 | JNK3 isoform 1 | SEQ ID NO: 1 |
| NM_001318069.1 | JNK3 isoform 1x | SEQ ID NO: 2 |
| NM_002753.4 | JNK3 isoform 2 | SEQ ID NO: 3 |
| NM_138980.3 | JNK3 isoform 3 | SEQ ID NO: 4 |
| NM_001318067.1 | JNK3 isoform 5 | SEQ ID NO: 5 |
| NM_001318068.1 | JNK3 isoform 6 | SEQ ID NO: 6 |

### Angiogenesis-Related Disorders

As used herein, the term “angiogenesis-related disorder” refers to a number of disease and disorders that are characterized by abnormal vasculature or poor vascularization. Non-limiting examples of angiogenesis-related disorders include: peripheral arterial disease (PAD), macular degeneration, retinopathy, stroke, diabetic limb ulcers, diabetic neuropathy, age-related blindness, chronic wounds, pressure ulcers, Alzheimer's disease, myocardial ischemia, cerebral ischemia, hepatic ischemia, limb ischemia, pulmonary ischemia, renal ischemia, testicular ischemia, intestinal type ischemia, or any organ ischemia.

### Methods of Treatment

The present methods include the use of JNK3 inhibitors, for treating angiogenesis-related disorders, e.g., peripheral arterial disease (PAD), in a subject who is in need of, or who has been determined to be in need of, such treatment. In some embodiments, the subject has peripheral arterial disease (PAD), macular degeneration, retinopathy, stroke, diabetic limb ulcers, diabetic neuropathy, age-related blindness, chronic wounds, pressure ulcers, Alzheimer's disease, myocardial ischemia, cerebral ischemia, hepatic ischemia, limb ischemia, pulmonary ischemia, renal ischemia, testicular ischemia, intestinal type ischemia, or any organ ischemia. Subjects can be diagnosed with an angiogenesis-related disorder using methods known in the art, e.g., be a healthcare provider.

The term “subject” refers to any mammal. In some embodiments, the subject or “subject suitable for treatment” may be a canine (e.g., a dog), feline (e.g., a cat), equine (e.g., a horse), bovine, ovine, caprine, porcine, primate, e.g., a simian (e.g., a monkey, e.g., a marmoset, or a baboon), or an ape (e.g., a gorilla, a chimpanzee, an orangutan, or a gibbon)) or a human; or rodent (e.g., a mouse, a guinea pig, a hamster, or a rat). In some embodiments, the subject or “subject suitable for treatment” may be a non-human mammal, especially mammals that are conventionally used as models for demonstrating therapeutic efficacy in humans (e.g., canine, lapine, murine, porcine, or primate animals) may be employed.

These methods can be used to treat a subject, e.g., a subject with an angiogenesis-related disorder (e.g., a subject with peripheral arterial disease), by administering to the subject a composition (e.g., as described herein) comprising a JNK3 inhibitor.

As used herein, treating includes “prophylactic treatment” which means reducing the incidence of or preventing (or reducing risk of) a sign or symptom of a disease in a patient at risk for the disease, and “therapeutic treatment”, which means reducing signs or symptoms of a disease, reducing progression of a disease, reducing severity of a disease, re-occurrence in a patient diagnosed with the disease.

As used herein in this context, to “treat” means to ameliorate at least one clinical parameter of the disease. In some embodiments, the parameter is blood flow or angiogenesis. In some embodiments, the parameter is wound healing or reduced signs of tissue ischemia such as hair regrowth, restoration of pulses, return of neurologic sensation, reduced circulation of ischemic markers such as lactate dehydrogenase (LDH) or creatine phosphokinase (CPK). In some embodiments, the parameter is the restoration of sight. In some embodiments, JNK3 inhibition is used to treat a subject that has, or is at risk of having a condition with inadequate blood flow recovery, such as diabetic wound healing, and medical conditions with virtually no effective treatments.

In some embodiments, treatment comprises the administration of specific JNK3 inhibitors. In some embodiments, the specific JNK3 inhibitor is a small molecule. In some embodiments, the JNK3 small molecule inhibitor is a N-(3-Cyano-4,5,6,7-tetrahydro-1-benzothien-2-yl)amide compound (e.g., JNK 5a or JNK 11a) (Angell et al., Bioorg. Med. Chem. Lett. 2007; 17(5): 1296-1301), an aminopyrazole inhibitor (e.g., SR-3576, SR-3737 (Kamenecka et al., J. Biol. Chem 2009; 284(19): 12853-61), 26n or 26k (Zheng et al., J. Med. Chem. 2014; 57(23): 10013-30)), a 6,7-dihydro-5H-pyrrolo[1,2-a]imidazole scaffold (Graczyk et al., Bioorg. Med. Chem. Lett. 2005; 15(21): 4666-70), a pyrazole inhibitor (e.g., 5r) (Jiang et al., Bioorg. Med. Chem. Lett. 2013; 23(9): 2683-7), 6-anilinoindazoles (Swahn et al., Bioorg. Med. Chem. Lett. 2005; 15(22): 5095-9) Quinazoline 13a (He et al., Bioorg. Med. Chem. Lett. 2011; 21(6): 1719-23), a 1-aryl-3,4-dihydroisoquinoline derivative (Christopher et al., Bioorg. Med. Chem. Lett. 2009; 19(8): 2230-4), 47 (Bowers et al., Bioorg Med. Chem. Lett. 2011; 21(6): 1838-43), 10 (Probst et al., Bioorg. Med. Chem. Lett. 2011; 21(1): 315-9), or 11H-Indeno[1,2-b]quinoxalin-11-one O-(2-furanylcarbonyl)oxime (e.g., IQ3) (Schepetkin et al., Mol. Pharmacol. 2012; 81(6): 832-45). In some embodiments, the JNK3 inhibitor is a peptide inhibitor. In some embodiments, the peptide inhibitor is peptide JNK3 inhibitor (Pan et al., PLoS One 2015; 10(4): e0119204). In some embodiments, the JNK3 small molecule inhibitor is not a broad-spectrum JNK inhibitor. In some embodiments, the JNK3 small molecule inhibitor is not SP600125 or AS601245. Further teachings of JNK3 inhibitors can be found in (Gehringer et al., Expert Opin. Ther. Pat. 2015; 25(8): 849-872; Messoussi et al., Chem Biol. 2014; 21(11): 1433-43; and Koch et al., J. Med. Chem. 2015; 58(1): 72-95), incorporated by reference herein.

Inhibitory nucleic acids have been employed as therapeutic moieties in the treatment of disease states in animals, including humans. Inhibitory nucleic acids can be useful therapeutic modalities that can be configured to be useful in treatment regimens for the treatments of cells, tissues and animals, especially humans.

For therapeutics, an animal, preferably a human, having, or suspected of having an angiogenesis-related disorder, or at increased risk of developing an angiogenesis-related disorder (e.g., by virtue of family history, genetic testing, or presence of other identified risk factor(s)), is treated by administering an inhibitory nucleic acid in accordance with this disclosure. For example, in one non-limiting embodiment, the methods comprise the step of administering to the animal in need of treatment a therapeutically effective amount of an inhibitory nucleic acid as described herein.

### Inhibitory Nucleic Acids

The methods described herein can include the administration of inhibitory nucleic acids that hybridize specifically to JNK3 to treat an angiogenesis-related disorder, e.g., peripheral arterial disease (PAD), macular degeneration, retinopathy, stroke, myocardial ischemia, cerebral ischemia, hepatic ischemia, limb ischemia, pulmonary ischemia, renal ischemia, testicular ischemia, intestinal type ischemia, or any organ ischemia.

A nucleic acid that “specifically” binds primarily to the target, i.e., to JNK3 but not to other non-target RNAs. The specificity of the nucleic acid interaction thus refers to its function (e.g., inhibiting JNK3) rather than its hybridization capacity. Oligos may exhibit nonspecific binding to other sites in the genome or other mRNAs, without interfering with binding of other regulatory proteins and without causing degradation of the non-specifically-bound RNA. Thus this nonspecific binding does not significantly affect function of other non-target RNAs and results in no significant adverse effects. Inhibitory agents useful in the methods of treatment described herein include inhibitory nucleic acid molecules that decrease the expression of activity of JNK3.

Inhibitory nucleic acids useful in the present methods and compositions include antisense oligonucleotides, siRNA compounds, single- or double-stranded RNA interference (RNAi) compounds, such as siRNA compounds, modified bases/locked nucleic acids (LNAs), and other oligomeric compounds, or oligonucleotide mimetics which hybridize to at least a portion of the target nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA), a small, temporal RNA (stRNA); or a short, hairpin RNA (shRNA); small RNA-induced gene activation (RNAa); small activating RNAs (saRNAs), or combinations thereof. See, e.g., WO 2010/040112.

In some embodiments, the inhibitory nucleic acids are 10 to 50, 13 to 50, or 13 to 30 nucleotides in length. One having ordinary skill in the art will appreciate that this embodies oligonucleotides having antisense portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin. In some embodiments, the oligonucleotides are 15 nucleotides in length. In some embodiments, the antisense or oligonucleotide compounds of the invention are 12 or 13 to 30 nucleotides in length. One having ordinary skill in the art will appreciate that this embodies inhibitory nucleic acids having antisense portions of 12, 13, 14, 15, 16, 17, 18, 19, 20, 21, 22, 23, 24, 25, 26, 27, 28, 29, or 30 nucleotides in length, or any range there within.

In some embodiments, the inhibitory nucleic acids are chimeric oligonucleotides that contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the target) and a region that is a substrate for enzymes capable of cleaving RNA: DNA or RNA: RNA hybrids. Chimeric inhibitory nucleic acids of the invention may be formed as composite structures of two or more oligonucleotides, modified oligonucleotides, oligonucleosides, and/or oligonucleotide mimetics as described above. Such compounds have also been referred to in the art as hybrids or gapmers. Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the inhibitory nucleic acid comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino, and 2′ O-methyl modifications on the ribose of pyrimidines, abasic residues, or an inverted base at the 3′ end of the RNA. Such modifications are routinely incorporated into oligonucleotides and these oligonucleotides have been shown to have a higher Tm (i.e., higher target binding affinity) than 2′-deoxyoligonucleotides against a given target.

A number of nucleotide and nucleoside modifications have been shown to make the oligonucleotide into which they are incorporated more resistant to nuclease digestion than the native oligodeoxynucleotide—the modified oligos survive intact for a longer time than unmodified oligonucleotides. Specific examples of modified oligonucleotides include those comprising modified backbones, for example, phosphorothioates, phosphotriesters, methyl phosphonates, short-chain alkyl or cycloalkyl intersugar linkages, or short-chain heteroatomic or heterocyclic intersugar linkages. Most preferred are oligonucleotides with phosphorothioate backbones and those with heteroatom backbones, particularly CH2-NH—O—CH2, CH, ˜N(CH3)˜O˜CH2 (known as a methylene(methylimino) or MMI backbone], CH2-O—N(CH3)-CH2, CH2-N(CH3)-N (CH3)-CH2 and O—N(CH3)-CH2-CH2 backbones, wherein the native phosphodiester backbone is represented as O— P—O—CH); amide backbones (see De Mesmaeker et al., Ace. Chem. Res. 28:366-374, 1995); morpholino backbone structures (see U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 254: 1497, 1991). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050 (each of which is incorporated by reference).

Morpholino-based oligomeric compounds are described in Braasch et al., Biochemistry 41(14):4503-4510, 2002; Genesis, volume 30, issue 3, 2001; Heasman, J., Dev. Biol., 243:209-214, 2002; Nasevicius et al., Nat. Genet. 26: 216-220, 2000; Lacerra et al., Proc. Natl. Acad. Sci. U.S.A. 97:9591-9596, 2000; and U.S. Pat. No. 5,034,506. Cyclohexenyl nucleic acid oligonucleotide mimetics are described in Wang et al., J. Am. Chem. Soc. 122, 8595-8602, 2000.

Modified oligonucleotide backbones that do not include a phosphorus atom therein have backbones that are formed by short-chain alkyl or cycloalkyl internucleoside linkages, mixed heteroatom and alkyl or cycloalkyl internucleoside linkages, or one or more short-chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264, 562; 5, 264,564; 5,405,938; 5,434,257; 5,466,677; 5,470,967; 5,489,677; 5,541,307; 5,561,225; 5,596, 086; 5,602,240; 5,610,289; 5,602,240; 5,608,046; 5,610,289; 5,618,704; 5,623, 070; 5,663,312; 5,633,360; 5,677,437; and 5,677,439 (each of which is herein incorporated by reference).

One or more substituted sugar moieties can also be included, e.g., one of the following at the 2′ position: OH, SH, SCH₃, F, OCN, OCH₃ OCH₃, OCH₃ O(CH₂)n CH₃, O(CH₂)n NH₂ or O(CH₂)n CH₃, where n is from 1 to about 10; Ci to C10 lower alkyl, alkoxyalkoxy, substituted lower alkyl, alkaryl or aralkyl; Cl; Br; CN; CF3; OCF3; O-, S-, or N-alkyl; O-, S-, or N-alkenyl; SOCH3; SO2 CH3; ONO2; NO2; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-0-CH₂CH₂OCH₃, also known as 2′-O-(2-methoxyethyl)] (Martin et al., HeIv. Chim. Acta 78:486, 1995). Other preferred modifications include 2′-methoxy (2′-0-CH₃), 2′-propoxy (2′-OCH₂ CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics, such as cyclobutyls in place of the pentofuranosyl group.

Inhibitory nucleic acids can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC, and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine, and 2,6-diaminopurine. See Kornberg, A., DNA Replication, W. H. Freeman & Co., San Francisco, 1980, pp 75-77; and Gebeyehu et al., Nucl. Acids Res. 15:4513, 1987. A “universal” base known in the art, e.g., inosine, can also be included. 5-Me-C substitutions have been shown to increase nucleic acid duplex stability by 0.6-1.2<0>C (Sanghvi, Y. S., in Crooke, S. T. and Lebleu, B., Eds., Antisense Research and Applications, CRC Press, Boca Raton, 1993, pp. 276-278) and are presently preferred base substitutions.

It is not necessary for all positions in a given oligonucleotide to be uniformly modified, and in fact more than one of the aforementioned modifications may be incorporated in a single oligonucleotide or even at within a single nucleoside within an oligonucleotide.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262, each of which is herein incorporated by reference. Further teaching of PNA compounds can be found in Nielsen et al, Science 254:1497-1500, 1991.

Inhibitory nucleic acids can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C), and uracil (U). Modified nucleobases comprise other synthetic and natural nucleobases, such as 5-methylcytosine (5-me-C), 5-hydroxymethyl cytosine, xanthine, hypoxanthine, 2-aminoadenine, 6-methyl, and other alkyl derivatives of adenine and guanine, 2-propyl and other alkyl derivatives of adenine and guanine, 2-thiouracil, 2-thiothymine, and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine, and 7-deazaadenine, and 3-deazaguanine and 3-deazaadenine.

Further, nucleobases comprise those disclosed in U.S. Pat. No. 3,687,808, those disclosed in ‘The Concise Encyclopedia of Polymer Science And Engineering’, pages 858-859, Kroschwitz, J. I., Ed. John Wiley & Sons, 1990, those disclosed by Englisch et al., Angewandle Chemie, International Edition’, 1991, 30, page 613, and those disclosed by Sanghvi, Y. S., Chapter 15, Antisense Research and Applications', pages 289-302, Crooke, S. T. and Lebleu, B. ea., CRC Press, 1993. Certain of these nucleobases are particularly useful for increasing the binding affinity of the oligomeric compounds of the invention. These include 5-substituted pyrimidines, 6-azapyrimidines, and N-2, N-6 and 0-6 substituted purines, comprising 2-aminopropyladenine, 5-propynyluracil, and 5-propynylcytosine. 5-methylcytosine substitutions have been shown to increase nucleic acid duplex stability by 0.6-1.2<0>C (Sanghvi, Y. S., Crooke, S. T. and Lebleu, B., Eds, ‘Antisense Research and Applications’, CRC Press, Boca Raton, 1993, pp. 276-278) and are presently preferred base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175, 273; 5, 367,066; 5,432,272; 5,457,187; 5,459,255; 5,484,908; 5,502,177; 5,525,711; 5,552,540; 5,587,469; 5,596,091; 5,614,617; 5,750,692, and 5,681,941 (each of which is herein incorporated by reference).

In some embodiments, the inhibitory nucleic acids are chemically linked to one or more moieties or conjugates that enhance the activity, cellular distribution, or cellular uptake of the oligonucleotide. Such moieties comprise but are not limited to, lipid moieties such as a cholesterol moiety (Letsinger et al., Proc. Natl. Acad. Sci. U.S.A. 86:6553-6556, 1989), cholic acid (Manoharan et al., Bioorg. Med. Chem. Lett. 4:1053-1060, 1994), a thioether, e.g., hexyl-S-tritylthiol (Manoharan et al, Ann. N. Y. Acad. Sci. 660:306-309, 1992; Manoharan et al., Bioorg. Med. Chem. Lett. 3:2765-2770, 1993), a thiocholesterol (Oberhauser et al., Nucl. Acids Res. 20, 533-538, 1992), an aliphatic chain, e.g., dodecandiol or undecyl residues (Kabanov et al., FEBS Lett. 259:327-330, 1990; Svinarchuk et al., Biochimie 75:49-54, 1993), a phospholipid, e.g., di-hexadecyl-rac-glycerol or triethylammonium 1,2-di-O-hexadecyl-rac-glycero-3-H-phosphonate (Manoharan et al., Tetrahedron Lett. 36:3651-3654, 1995; Shea et al., Nucl. Acids Res. 18:3777-3783, 1990), a polyamine or a polyethylene glycol chain (Mancharan et al., Nucleosides & Nucleotides 14:969-973, 1995), or adamantane acetic acid (Manoharan et al., Tetrahedron Lett. 36:3651-3654, 1995), a palmityl moiety (Mishra et al., Biochim. Biophys. Acta 1264: 229-237, 1995), or an octadecylamine or hexylamino-carbonyl-t oxycholesterol moiety (Crooke et al., J. Pharmacol. Exp. Ther. 277:923-937, 1996). See also U.S. Pat. Nos. 4,828,979; 4,948,882; 5,218,105; 5,525,465; 5,541,313; 5,545,730; 5,552, 538; 5,578,717, 5,580,731; 5,580,731; 5,591,584; 5,109,124; 5,118,802; 5,138,045; 5,414,077; 5,486, 603; 5,512,439; 5,578,718; 5,608,046; 4,587,044; 4,605,735; 4,667,025; 4,762, 779; 4,789,737; 4,824,941; 4,835,263; 4,876,335; 4,904,582; 4,958,013; 5,082, 830; 5,112,963; 5,214,136; 5,082,830; 5,112,963; 5,214,136; 5, 245,022; 5,254,469; 5,258,506; 5,262,536; 5,272,250; 5,292,873; 5,317,098; 5,371,241, 5,391, 723; 5,416,203, 5,451,463; 5,510,475; 5,512,667; 5,514,785; 5, 565,552; 5,567,810; 5,574,142; 5,585,481; 5,587,371; 5,595,726; 5,597,696; 5,599,923; 5,599, 928 and 5,688,941 (each of which is herein incorporated by reference).

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism, or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941 (each of which is incorporated by reference).

The inhibitory nucleic acids useful in the present methods are sufficiently complementary to the target mRNA, i.e., hybridize sufficiently well and with sufficient specificity, to give the desired effect. “Complementary” refers to the capacity for pairing, through hydrogen bonding, between two sequences comprising naturally or non-naturally occurring bases or analogs thereof. For example, if a base at one position of an inhibitory nucleic acid is capable of hydrogen bonding with a base at the corresponding position of a mRNA, then the bases are considered to be complementary to each other at that position. In some embodiments, 100% complementarity is not required. In some embodiments, 100% complementarity is required. Routine methods can be used to design an inhibitory nucleic acid that binds to the target sequence with sufficient specificity.

While the specific sequences of certain exemplary target segments are set forth herein, one of skill in the art will recognize that these serve to illustrate and describe particular embodiments within the scope of the present invention. Additional target segments are readily identifiable by one having ordinary skill in the art in view of this disclosure. Target segments of 5, 6, 7, 8, 9, 10 or more nucleotides in length comprising a stretch of at least five (5) consecutive nucleotides within the seed sequence, or immediately adjacent thereto, are considered to be suitable for targeting as well. In some embodiments, target segments can include sequences that comprise at least the 5 consecutive nucleotides from the 5′-terminus of one of the seed sequence (the remaining nucleotides being a consecutive stretch of the same RNA beginning immediately upstream of the 5′-terminus of the seed sequence and continuing until the inhibitory nucleic acid contains about 5 to about 30 nucleotides). In some embodiments, target segments are represented by RNA sequences that comprise at least the 5 consecutive nucleotides from the 3′-terminus of one of the seed sequence (the remaining nucleotides being a consecutive stretch of the same mRNA beginning immediately downstream of the 3′-terminus of the target segment and continuing until the inhibitory nucleic acid contains about 5 to about 30 nucleotides). One having skill in the art armed with the sequences provided herein will be able, without undue experimentation, to identify further preferred regions to target. In some embodiments, an inhibitory nucleic acid contains a sequence that is complementary to at least 5, 6, 7, 8, 9, 10, 11, 12, 13, 14, 15, 16, 17, 18, 19, 20, 21, 22, 23, 24, or 25 contiguous nucleotides present in the target (e.g., the target mRNA).

Once one or more target regions, segments or sites have been identified, inhibitory nucleic acid compounds are chosen that are sufficiently complementary to the target, i.e., that hybridize sufficiently well and with sufficient specificity (i.e., do not substantially bind to other non-target RNAs), to give the desired effect.

In the context of this invention, hybridization means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as used herein, refers to the capacity for precise pairing between two nucleotides. For example, if a nucleotide at a certain position of an oligonucleotide is capable of hydrogen bonding with a nucleotide at the same position of an mRNA molecule, then the inhibitory nucleic acid and the mRNA are considered to be complementary to each other at that position. The inhibitory nucleic acids and the mRNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the inhibitory nucleic acid and the mRNA target. For example, if a base at one position of an inhibitory nucleic acid is capable of hydrogen bonding with a base at the corresponding position of a mRNA, then the bases are considered to be complementary to each other at that position. 100% complementarity is not required.

It is understood in the art that a complementary nucleic acid sequence need not be 100% complementary to that of its target nucleic acid to be specifically hybridizable. A complementary nucleic acid sequence for purposes of the present methods is specifically hybridisable when binding of the sequence to the target mRNA molecule interferes with the normal function of the target mRNA to cause a loss of expression or activity, and there is a sufficient degree of complementarity to avoid non-specific binding of the sequence to non-target RNA sequences under conditions in which specific binding is desired, e.g., under physiological conditions in the case of in vivo assays or therapeutic treatment, and in the case of in vitro assays, under conditions in which the assays are performed under suitable conditions of stringency. For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci. U.S.A. 72:3961, 1975); Ausubel et al., (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

In general, the inhibitory nucleic acids useful in the methods described herein have at least 80% sequence complementarity to a target region within the target nucleic acid, e.g., 90%, 95%, or 100% sequence complementarity to the target region within a mRNA. For example, an antisense compound in which 18 of 20 nucleobases of the antisense oligonucleotide are complementary, and would therefore specifically hybridize, to a target region would represent 90 percent complementarity. Percent complementarity of an inhibitory nucleic acid with a region of a target nucleic acid can be determined routinely using basic local alignment search tools (BLAST programs) (Altschul et al., J. Mol. Biol. 215:403-410, 1990; Zhang and Madden, Genome Res. 7:649-656, 1997). Antisense and other compounds of the invention that hybridize to a mRNA are identified through routine experimentation. In general, the inhibitory nucleic acids must retain specificity for their target, i.e., must not directly bind to, or directly significantly affect expression levels of, transcripts other than the intended target.

For further disclosure regarding inhibitory nucleic acids, please see US2010/0317718 (antisense oligos); US2010/0249052 (double-stranded ribonucleic acid (dsRNA)); US2009/0181914 and US2010/0234451 (LNAs); US2007/0191294 (siRNA analogues); US2008/0249039 (modified siRNA); and WO2010/129746 and WO2010/040112 (inhibitory nucleic acids).

Antisense

Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under stringent conditions to the target mRNA. Thus, oligonucleotides are chosen that are sufficiently complementary to the target, i.e., that hybridize sufficiently well and with sufficient specificity, to give the desired effect.

Modified Bases/Locked Nucleic Acids (LNAs)

In some embodiments, the inhibitory nucleic acids used in the methods described herein comprise one or more modified bonds or bases. Modified bases include phosphorothioate, methylphosphonate, peptide nucleic acids, or locked nucleic acid (LNA) molecules. Preferably, the modified nucleotides are locked nucleic acid molecules, including [alpha]-L-LNAs. LNAs comprise ribonucleic acid analogues wherein the ribose ring is “locked” by a methylene bridge between the 2′-oxgygen and the 4′-carbon—i.e., oligonucleotides containing at least one LNA monomer, that is, one 2′-O,4′-C-methylene-β-D-ribofuranosyl nucleotide. LNA bases form standard Watson-Crick base pairs but the locked configuration increases the rate and stability of the base pairing reaction (Jepsen et al., Oligonucleotides 14:130-146, 2004). LNAs also have increased affinity to base pair with RNA as compared to DNA. These properties render LNAs especially useful as probes for fluorescence in situ hybridization (FISH) and comparative genomic hybridization, as antisense oligonucleotides to target mRNAs as described herein.

The LNA molecules can include molecules comprising 10-30, e.g., 12-24, e.g., 12, 13, 14, 15, 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the mRNA. The LNA molecules can be chemically synthesized using methods known in the art.

The LNA molecules can be designed using any method known in the art; a number of algorithms are known, and are commercially available (e.g., on the internet, for example at exiqon.com). See, e.g., You et al., Nuc. Acids. Res. 34: e60, 2006; McTigue et al., Biochemistry 43:5388-405, 2004; and Levin et al., Nucl. Acids. Res. 34: e142, 2006. For example, “gene walk” methods, similar to those used to design antisense oligos, can be used to optimize the inhibitory activity of the LNA; for example, a series of oligonucleotides of 10-30 nucleotides spanning the length of a target mRNA can be prepared, followed by testing for activity. Optionally, gaps, e.g., of 5-10 nucleotides or more, can be left between the LNAs to reduce the number of oligonucleotides synthesized and tested. GC content is preferably between about 30-60%. General guidelines for designing LNAs are known in the art; for example, LNA sequences will bind very tightly to other LNA sequences, so it is preferable to avoid significant complementarity within an LNA. Contiguous runs of three or more Gs or Cs, or more than four LNA residues, should be avoided where possible (for example, it may not be possible with very short (e.g., about 9-10 nt) oligonucleotides). In some embodiments, the LNAs are xylo-LNAs.

For additional information regarding LNAs see U.S. Pat. Nos. 6,268,490; 6,734,291; 6,770,748; 6,794,499; 7,034,133; 7,053,207; 7,060,809; 7,084,125; and 7,572,582; and U.S. Pre-Grant Pub. Nos. 2010/0267018; 2010/0261175; and 2010/0035968; Koshkin et al., Tetrahedron 54:3607-3630, 1998; Obika et al., Tetrahedron Lett. 39:5401-5404, 1998; Jepsen et al., Oligonucleotides 14:130-146, 2004; Kauppinen et al., Drug Disc. Today 2(3):287-290, 2005; and Ponting et al., Cell 136(4):629-641, 2009, and references cited therein.

See also U.S. Ser. No. 61/412,862, which is incorporated by reference herein in its entirety.

siRNA

In some embodiments, the nucleic acid sequence that is complementary to a target mRNA can be an interfering RNA, including but not limited to a small interfering RNA (“siRNA”) or a small hairpin RNA (“shRNA”). Methods for constructing interfering RNAs are well known in the art. For example, the interfering RNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure); the antisense strand comprises nucleotide sequence that is complementary to a nucleotide sequence in a target nucleic acid molecule or a portion thereof (i.e., an undesired gene) and the sense strand comprises nucleotide sequence corresponding to the target nucleic acid sequence or a portion thereof. Alternatively, interfering RNA is assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to the target nucleic acid sequence or a portion thereof. The interfering can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNA interference.

In some embodiments, the interfering RNA coding region encodes a self-complementary RNA molecule having a sense region, an antisense region and a loop region. Such an RNA molecule when expressed desirably forms a “hairpin” structure, and is referred to herein as an “shRNA.” The loop region is generally between about 2 and about 10 nucleotides in length. In some embodiments, the loop region is from about 6 to about 9 nucleotides in length. In some embodiments, the sense region and the antisense region are between about 15 and about 20 nucleotides in length. Following post-transcriptional processing, the small hairpin RNA is converted into a siRNA by a cleavage event mediated by the enzyme Dicer, which is a member of the RNase III family. The siRNA is then capable of inhibiting the expression of a gene with which it shares homology. For details, see Brummelkamp et al., Science 296:550-553, 2002; Lee et al., Nature Biotechnol., 20, 500-505, 2002; Miyagishi and Taira, Nature Biotechnol. 20:497-500, 2002; Paddison et al., Genes & Dev. 16:948-958, 2002; Paul, Nature Biotechnol. 20, 505-508, 2002; Sui, Proc. Natl. Acad. Sci. U.S.A., 99(6):5515-5520, 2002; Yu et al., Proc. Natl. Acad. Sci. U.S.A. 99:6047-6052, 2002.

The target RNA cleavage reaction guided by siRNAs is highly sequence specific. In general, siRNA containing a nucleotide sequences identical to a portion of the target nucleic acid (i.e., a target region comprising the seed sequence of a target mRNA) are preferred for inhibition. However, 100% sequence identity between the siRNA and the target gene is not required to practice the present invention. Thus the invention has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Alternatively, siRNA sequences with nucleotide analog substitutions or insertions can be effective for inhibition. In general, the siRNAs must retain specificity for their target, i.e., must not directly bind to, or directly significantly affect expression levels of, transcripts other than the intended target.

Ribozymes

Trans-cleaving enzymatic nucleic acid molecules can also be used; they have shown promise as therapeutic agents for human disease (Usman & McSwiggen, Ann. Rep. Med. Chem. 30:285-294, 1995; Christoffersen and Marr, J. Med. Chem. 38:2023-2037, 1995). Enzymatic nucleic acid molecules can be designed to cleave specific mRNA targets within the background of cellular RNA. Such a cleavage event renders the mRNA non-functional.

In general, enzymatic nucleic acids with RNA cleaving activity act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA will destroy its activity. After an enzymatic nucleic acid has bound and cleaved its RNA target, it is released from that RNA to search for another target and can repeatedly bind and cleave new targets.

Several approaches such as in vitro selection (evolution) strategies (Orgel, Proc. R. Soc. London, B 205:435, 1979) have been used to evolve new nucleic acid catalysts capable of catalyzing a variety of reactions, such as cleavage and ligation of phosphodiester linkages and amide linkages, (Joyce, Gene, 82, 83-87, 1989; Beaudry et al., Science 257, 635-641, 1992; Joyce, Scientific American 267, 90-97, 1992; Breaker et al., TIBTECH 12:268, 1994; Bartel et al., Science 261:1411-1418, 1993; Szostak, TIBS 17, 89-93, 1993; Kumar et al., FASEB J., 9:1183, 1995; Breaker, Curr. Op. Biotech., 1:442, 1996). The development of ribozymes that are optimal for catalytic activity would contribute significantly to any strategy that employs RNA-cleaving ribozymes for the purpose of regulating gene expression. The hammerhead ribozyme, for example, functions with a catalytic rate (kcat) of about 1 min⁻¹ in the presence of saturating (10 rnM) concentrations of Mg²⁺ cofactor. An artificial “RNA ligase” ribozyme has been shown to catalyze the corresponding self-modification reaction with a rate of about 100 min⁻¹. In addition, it is known that certain modified hammerhead ribozymes that have substrate binding arms made of DNA catalyze RNA cleavage with multiple turn-over rates that approach 100 min⁻¹.

Making and Using Inhibitory Nucleic Acids and Sense Nucleic Acids

The nucleic acid sequences used to practice the methods described herein, whether RNA, cDNA, genomic DNA, vectors, viruses or hybrids thereof, can be isolated from a variety of sources, genetically engineered, amplified, and/or expressed/generated recombinantly. Recombinant nucleic acid sequences can be individually isolated or cloned and tested for a desired activity. Any recombinant expression system can be used, including e.g., in vitro, bacterial, fungal, mammalian, yeast, insect, or plant cell expression systems.

Nucleic acid sequences of the invention (e.g., any of the inhibitory nucleic acids described herein) can be inserted into delivery vectors and expressed from transcription units within the vectors. The recombinant vectors can be DNA plasmids or viral vectors. Generation of the vector construct can be accomplished using any suitable genetic engineering techniques well known in the art, including, without limitation, the standard techniques of PCR, oligonucleotide synthesis, restriction endonuclease digestion, ligation, transformation, plasmid purification, and DNA sequencing, for example as described in Sambrook et al., Molecular Cloning: A Laboratory Manual. (1989)), Coffin et al., (Retroviruses. (1997)) and “RNA Viruses: A Practical Approach” (Alan J. Cann, Ed., Oxford University Press, (2000)).

As will be apparent to one of ordinary skill in the art, a variety of suitable vectors are available for transferring nucleic acids of the invention into cells. The selection of an appropriate vector to deliver nucleic acids and optimization of the conditions for insertion of the selected expression vector into the cell, are within the scope of one of ordinary skill in the art without the need for undue experimentation. Viral vectors comprise a nucleotide sequence having sequences for the production of recombinant virus in a packaging cell. Viral vectors expressing nucleic acids of the invention can be constructed based on viral backbones including, but not limited to, a retrovirus, lentivirus, herpes virus, adenovirus, adeno-associated virus, pox virus, or alphavirus. The recombinant vectors (e.g., viral vectors) capable of expressing the nucleic acids of the invention can be delivered as described herein, and persist in target cells (e.g., stable transformants). For example, such recombinant vectors (e.g., a recombinant vector that results in the expression of an antisense oligomer that is complementary to JNK3) can be administered into (e.g., injection or infusion into) a subject (e.g., intracranial injection, intraparenchymal injection, intraventricular injection, and intrathecal injection, see, e.g., Bergen et al., Pharmaceutical Res. 25:983-998, 2007). A number of exemplary recombinant viral vectors that can be used to express any of the nucleic acids described herein are also described in Bergen et al., (supra). Additional examples of recombinant viral vectors are known in the art.

The nucleic acids provided herein (e.g., the inhibitory nucleic acids) can be further be complexed with one or more cationic polymers (e.g., poly-L-lysine and poly(ethylenimine), cationic lipids (e.g., 1,2-dioleoyl-3-trimethylammonium propone (DOTAP), N-methyl-4-(dioleyl)methylpyridinium, and 3β-[N—(N′,N′-dimethylaminoethane)-carbamoyl] cholesterol), and/or nanoparticles (e.g., cationic polybutyl cyanoacrylate nanoparticles, silica nanoparticles, or polyethylene glycol-based nanoparticles) prior to administration to the subject (e.g., injection or infusion into the cerebrospinal fluid of the subject). Additional examples of cationic polymers, cationic lipids, and nanoparticles for the therapeutic delivery of nucleic acids are known in the art. The therapeutic delivery of nucleic acids has also been shown to be achieved following intrathecal injection of polyethyleneimine/DNA complexes (Wang et al., Mol. Ther. 12:314-320, 2005). The methods for delivery of nucleic acids described herein are non-limiting. Additional methods for the therapeutic delivery of nucleic acids to a subject are known in the art.

In some embodiments, the inhibitory nucleic acids (e.g., one or more inhibitory nucleic acids targeting JNK3) can be administered systemically (e.g., intravenously, intaarterially, intramuscularly, subcutaneously, or intraperitoneally) or intrathecally (e.g., epidural administration). In some embodiments, the inhibitory nucleic acid is administered in a composition (e.g., complexed with) one or more cationic lipids. Non-limiting examples of cationic lipids that can be used to administer one or more inhibitory nucleic acids (e.g., any of the inhibitory nucleic acids described herein) include: Lipofectamine, the cationic lipid molecules described in WO 97/045069, and U.S. Patent Application Publication Nos. 2012/0021044, 2012/0015865, 2011/0305769, 2011/0262527, 2011/0229581, 2010/0305198, 2010/0203112, and 2010/0104629 (each of which is herein incorporated by reference). Nucleic acid sequences used to practice this invention can be synthesized in vitro by well-known chemical synthesis techniques, as described in, e.g., Adams, J. Am. Chem. Soc. 105:661, 1983; Belousov, Nucleic Acids Res. 25:3440-3444, 1997; Frenkel, Free Radic. Biol. Med. 19:373-380, 1995; Blommers, Biochemistry 33:7886-7896, 1994; Narang, Meth. Enzymol. 68:90, 1994; Brown, Meth. Enzymol. 68:109, 1979; Beaucage, Tetra. Lett. 22:1859, 1981; and U.S. Pat. No. 4,458,066.

Nucleic acid sequences of the invention can be stabilized against nucleolytic degradation such as by the incorporation of a modification, e.g., a nucleotide modification. For example, nucleic acid sequences of the invention include a phosphorothioate at least the first, second, or third internucleotide linkage at the 5′ or 3′ end of the nucleotide sequence. As another example, the nucleic acid sequence can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). As another example, the nucleic acid sequence can include at least one 2′-O-methyl-modified nucleotide, and in some embodiments, all of the nucleotides include a 2′-O-methyl modification. In some embodiments, the nucleic acids are “locked,” i.e., comprise nucleic acid analogues in which the ribose ring is “locked” by a methylene bridge connecting the 2′-O atom and the 4′-C atom (see, e.g., Kaupinnen et al., Drug Disc. Today 2(3):287-290, 2005; Koshkin et al., J. Am. Chem. Soc., 120(50):13252-13253, 1998). For additional modifications see US 2010/0004320, US 2009/0298916, and US 2009/0143326 (each of which is incorporated by reference).

Techniques for the manipulation of nucleic acids used to practice this invention, such as, e.g., subcloning, labeling probes (e.g., random-primer labeling using Klenow polymerase, nick translation, amplification), sequencing, hybridization, and the like are well described in the scientific and patent literature, see, e.g., Sambrook et al., Molecular Cloning; A Laboratory Manual 3d ed. (2001); Current Protocols in Molecular Biology, Ausubel et al., Eds. (John Wiley & Sons, Inc., New York 2010); Kriegler, Gene Transfer and Expression: A Laboratory Manual (1990); Laboratory Techniques In Biochemistry And Molecular Biology: Hybridization With Nucleic Acid Probes, Part I. Theory and Nucleic Acid Preparation, Tijssen, Ed. Elsevier, N.Y. (1993).

Various molecular biology techniques are well-known in the art to introduce a mutation(s) or a deletion(s) into an endogenous locus. In some embodiments of any of the methods described herein, JNK3 expression may be altered or lost by using techniques known in the art to disrupt the endogenous JNK3 gene locus. Non-limiting examples of such techniques include: site-directed mutagenesis, CRISPR (e.g., CRISPR/Cas9-induced knock-in mutations, or CRISPR/Cas9-induced knock-out mutations), or TALENs. Skilled practitioners will appreciate that the nucleic acids and expression vectors described herein can be introduced into any subject (e.g., introduced into any cell of a subject), for example, by lipofection, or CRISPR, and can be stably integrated into an endogenous gene locus.

As used herein, “Clustered regularly interspaced short palindromic repeats” or “CRISPR” refers to a two component ribonucleoprotein complex with a Cas9 nuclease and a guide RNA. Bacteria and archaea used this system to detect and silence foreign nucleic acids in a sequence-specific manner (Jinek et al., Science 2012; 337(6096): 816-21). Methods of how to make and use CRISPR/Cas9 constructs are widely available and known by those skilled in the art, e.g., Cho et al., Nature Biotech. 2013; 31: 230-232; Cong et al., Science 2013; 339(6121): 819-23; Hwang et al., Nature Biotech. 2013; 31(3): 227-9; Jiang et al., Nature Biotech. 2013; 31(3): 233-9; and Mali et al., Science 2013; 339(6121): 823-6.

### Pharmaceutical Compositions

The methods described herein can include the administration of pharmaceutical compositions and formulations comprising any one or more (e.g., two, three, four, or five) of the inhibitory nucleic acids targeting JNK3 as active ingredients, e.g., in some embodiments as the sole active ingredient. In some embodiments, the compositions are formulated with a pharmaceutically acceptable carrier. The pharmaceutical compositions and formulations can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21st ed., 2005.

The inhibitory nucleic acids can be administered alone or as a component of a pharmaceutical formulation (composition). The compounds may be formulated for administration, in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives, and antioxidants can also be present in the compositions. In some embodiments, one or more cationic lipids, cationic polymers, or nanoparticles can be included in compositions containing the one or more inhibitory nucleic acids (e.g., compositions containing one or more inhibitory nucleic acids targeting JNK3).

Formulations of the compositions of the invention include those suitable for intradermal, inhalation, oral/nasal, topical, parenteral, rectal, and/or intravaginal administration. The formulations may conveniently be presented in unit dosage form and may be prepared by any methods well known in the art of pharmacy. The amount of active ingredient (e.g., nucleic acid sequences of this invention) which can be combined with a carrier material to produce a single dosage form will vary depending upon the host being treated, the particular mode of administration, e.g., intradermal or inhalation. The amount of active ingredient which can be combined with a carrier material to produce a single dosage form will generally be that amount of the compound which produces a therapeutic effect.

Pharmaceutical formulations of this invention can be prepared according to any method known to the art for the manufacture of pharmaceuticals. Such drugs can contain sweetening agents, flavoring agents, coloring agents, and preserving agents. A formulation can be admixtured with nontoxic pharmaceutically acceptable excipients which are suitable for manufacture. Formulations may comprise one or more diluents, emulsifiers, preservatives, buffers, excipients, etc., and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., inhibitory nucleic acids or sense nucleic acids described herein) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth, and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long-chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents, and one or more sweetening agents, such as sucrose, aspartame, or saccharin. Formulations can be adjusted for osmolarity.

In some embodiments, oil-based pharmaceuticals are used for administration of nucleic acid sequences of the invention. Oil-based suspensions can be formulated by suspending an active agent in a vegetable oil, such as arachis oil, olive oil, sesame oil, or coconut oil, or in a mineral oil such as liquid paraffin; or a mixture of these. See e.g., U.S. Pat. No. 5,716,928, describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin, or cetyl alcohol. Sweetening agents can be added to provide a palatable oral preparation, such as glycerol, sorbitol, or sucrose. These formulations can be preserved by the addition of an antioxidant such as ascorbic acid. As an example of an injectable oil vehicle, see Minto, J. Pharmacol. Exp. Ther. 281:93-102, 1997.

Pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters, or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In alternative embodiments, these injectable oil-in-water emulsions of the invention comprise a paraffin oil, a sorbitan monooleate, an ethoxylated sorbitan monooleate, and/or an ethoxylated sorbitan trioleate.

The pharmaceutical compounds can also be administered by in intranasal, intraocular and intravaginal routes including suppositories, insufflation, powders and aerosol formulations (for examples of steroid inhalants, see e.g., Rohatagi, J. Clin. Pharmacol. 35:1187-1193, 1995; Tjwa, Ann. Allergy Asthma Immunol. 75:107-111, 1995). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In some embodiments, the pharmaceutical compounds can be delivered transdermally, by a topical route, formulated as applicator sticks, solutions, suspensions, emulsions, gels, creams, ointments, pastes, jellies, paints, powders, and aerosols.

In some embodiments, the pharmaceutical compounds can also be delivered as microspheres for slow release in the body. For example, microspheres can be administered via intradermal injection of drug which slowly release subcutaneously; see Rao, J. Biomater Sci. Polym. Ed. 7:623-645, 1995; as biodegradable and injectable gel formulations, see, e.g., Gao, Pharm. Res. 12:857-863, 1995; or, as microspheres for oral administration, see, e.g., Eyles, J. Pharm. Pharmacol. 49:669-674, 1997.

In some embodiments, the pharmaceutical compounds can be parenterally administered, such as by intravenous (IV) administration or administration into a body cavity, a lumen of an organ, or into the cranium (e.g., intracranial injection or infusion) or the cerebrospinal fluid of a subject. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose, any bland fixed oil can be employed including synthetic mono- or diglycerides. In addition, fatty acids, such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate, and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

In some embodiments, the pharmaceutical compounds and formulations can be lyophilized. Stable lyophilized formulations comprising an inhibitory nucleic acid or a sense nucleic acid can be made by lyophilizing a solution comprising a pharmaceutical of the invention and a bulking agent, e.g., mannitol, trehalose, raffinose, and sucrose, or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5, but less than 6.5. See, e.g., US2004/0028670.

The compositions and formulations can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989.

The formulations of the invention can be administered for prophylactic and/or therapeutic treatments. In some embodiments, for therapeutic applications, compositions are administered to a subject who is at risk of or has a disorder described herein, in an amount sufficient to cure, alleviate or partially arrest the clinical manifestations of the disorder or its complications; this can be called a therapeutically effective amount. For example, in some embodiments, pharmaceutical compositions of the invention are administered in an amount sufficient to reduce the number of symptoms or reduce the severity, duration, or frequency of one or more symptoms of a neurodegenerative disorder in a subject.

The amount of pharmaceutical composition adequate to accomplish this is a therapeutically effective dose. The dosage schedule and amounts effective for this use, i.e., the dosing regimen, will depend upon a variety of factors, including the stage of the disease or condition, the severity of the disease or condition, the general state of the patient's health, the patient's physical status, age, and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones, J. Steroid Biochem. Mol. Biol. 58:611-617, 1996; Groning, Pharmazie 51:337-341, 1996; Fotherby, Contraception 54:59-69, 1996; Johnson, J. Pharm. Sci. 84:1144-1146, 1995; Rohatagi, Pharmazie 50:610-613, 1995; Brophy, Eur. J. Clin. Pharmacol. 24:103-108, 1983; Remington: The Science and Practice of Pharmacy, 21st ed., 2005). The state of the art allows the clinician to determine the dosage regimen for each individual patient, active agent, and disease or condition treated. Guidelines provided for similar compositions used as pharmaceuticals can be used as guidance to determine the dosage regiment, i.e., dose schedule and dosage levels, administered practicing the methods of the invention are correct and appropriate.

Single or multiple administrations of formulations can be given depending on for example: the dosage and frequency as required and tolerated by the patient, and the like. The formulations should provide a sufficient quantity of active agent to effectively treat, prevent or ameliorate conditions, diseases, or symptoms.

In alternative embodiments, pharmaceutical formulations for oral administration are in a daily amount of between about 1 to 100 or more mg per kilogram of body weight per day. Lower dosages can be used, in contrast to administration orally, into the blood stream, into a body cavity or into a lumen of an organ. Substantially higher dosages can be used in topical or oral administration or administering by powders, spray, or inhalation. Actual methods for preparing parenterally or non-parenterally administrable formulations will be known or apparent to those skilled in the art and are described in more detail in such publications as Remington: The Science and Practice of Pharmacy, 21st ed., 2005.

### EXAMPLES

The invention is further described in the following examples, which do not limit the scope of the invention described in the claims.

### Example 1—Materials and Methods

Methods

### Animals and Hind Limb Ischemia Model

C57BL/6J strain mice were obtained from The Jackson Laboratories. Mice with Jnk3 gene disruptions have been described previously.

The mice were housed in a facility accredited by the American Association for Laboratory Animal Care. All animal studies were approved by the Institutional Animal Care and Use Committee of the University of Massachusetts Medical School.

Heterozygous JNK3-null animals on the C57 background were bred to homogeneity with age-matched controls (Yang et al., Nature 1997; 289:864-870). Male mice at 8 to 12 weeks of age were anesthetized with intraperitoneal injection of combination of 100 mg/kg ketamine hydrochloride and 5 mg/kg xylazine (Webster Veterinary, Devens, Mass.) before surgery. Unilateral hind limb ischemia in the right leg was introduced in the mice in Craige et al., Circulation 2011; 124: 731-740. In selected experiments, unilateral hind limb ischemia was performed 3 days after of a single dose of 30 μL (2.0×108 pfu) of adenoviral vectors encoding Creb1 (Vector Biolabs #1363), or GFP (a kind gift from Cooper lab) was injected into gastrocnemius muscles. Hind limb tissue perfusion was assessed with either moorLDI2-IR laser-Doppler imaging system or moorFLPI-2 blood flow imager (Moor Instruments, Devon, UK). Blood flow images were obtained under conditions of constant body temperature (36a1.0° C.) and average hind limb blood flow was expressed as the ratio of ischemic to nonischemic foot flow to account for minor variations in imaging conditions.

### Gene Painting

Gene painting in the right leg of mice were introduced as described in Igarashi et al., Circulation 2012; 125: 216-225. In brief gene painting with a solution containing 2 g/L poloxamer-F127 and 2×108 pfu/ml of the control GFP and shRNA for JNK3 adenovirus (Vector Biolabs #shADV-264201) was used as indicated.

### Cell Culture and Transfections

Mus musculus brain neuroblastoma cell line Neuro-2a (#CCL-131) were purchased from ATCC and cultured in Dulbecco's modified Eagle's medium supplemented with 10% fetal bovine serum, 100 units/ml penicillin, 100 μg/ml streptomycin, and 2 mM L-glutamine (Invitrogen).

Transfection assays were performed using 100 nM small interfering RNA oligonucleotides ON-TARGET plus SMART pool for control (D-001810-10), Jnk3 (MAPK10) (L-045023-00), Creb1 (L-040959-01), Sirtuin1 (L-049440-00) and Nrf1 (L-041037-01) (Thermo Scientific Dharmacon, Lafayette, Colo.) in DharmaFECT 3 reagent with for 6-8 hours in optimum (Invitrogen) as described in Kant et al., Genes Dev. 2011; 25: 2069-2078. Then media was changed to Dulbecco's modified Eagle's medium supplemented with 10% fetal bovine serum, 100 units/ml penicillin, 100 μg/ml streptomycin, and 2 mM L-glutamine (Invitrogen). After 48-72 hours of siRNA treatment cells were exposed with low serum (0.5%) and hypoxia (1% oxygen) for one hour in hypoxia chamber (Billups-Rothenberg Inc.). cAMP response element (CRE) luciferase assay (Qiagen # CCS-002L) was performed according to manufacture instructions.

### RNA Preparation and Quantitative Real-Time Polymerase Chain Reaction

Total RNA was extracted from cells and tissues with the RNeasy Mini Kit (Qiagen) or TRIzol reagent (Invitrogen), and 1 μg of total RNA was reverse transcribed with oligo(dT) primers for cDNA synthesis. The expression of mRNA was examined by quantitative PCR analysis using a QuantStudio™ 6 Flex Real-Time PCR System (Applied Biosystems). TaqmanC assays were used to quantitate Pdgfb (Mm00440677_m1), Tgfb3 (Mm00436960_m1), Tfb1m (Mm00524825_m1), Tfam (Mm00447485_m1), Pgf (Mm00435613_m1), Npy (Mm03048253_m1), Npb (Mm00462726_m1), Jnk3 (Mm00436518_m1), Hbegf (Mm00439306_m1), Creb1 (Mm00501607_m1), Nrf1 (Mm01135606_m1), Crtc1 (Mm01349190_m1), Ucp3 (Mm00494077_m1), Hprt (Mm00446968_m1) and Gapdh (4352339E-0904021) mRNA (Applied Biosystems). The 2-ΔΔCT method is used for relative quantification of gene as described in Livak et al., Methods 2001; 25:402-408 and Rao et al., Biostat. Bioinforma Biomath 2013; 3: 71-75. Reference genes of Hprt and Gapdh have been used to normalize the PCRs in each sample.

### Antibodies and Immunoblot Analysis

Cell extracts were prepared using Triton lysis buffer [20 mM Tris (pH 7.4), 1% Triton X-100, 10% glycerol, 137 mM NaCl, 2 mM EDTA, 25 mM b-glycerophosphate, 1 mM sodium orthovanadate, 1 mM phenylmethylsulfonyl fluoride, and 10 μg/mL of aprotinin and leupeptin]. Protein extracts (50 μg of protein) in DTT-containing SDS sample buffer were separated in 10% or 12% SDS-polyacrylamide gels and transferred to Hybond ECL nitrocellulose membranes (GE Healthcare, Piscataway, N.J.). Immunecomplexes were detected by Amersham™ Imager 600 using Immobilon Western HRP Substrate (EMD Millipore). Primary antibodies were obtained from Cell Signaling (phospho-Creb1 #9198, Creb1 #9197, #9104 phospho-Sirtuin1 #2314 and Sirtuin1), EMD Millipore (Acetyl Lysine clone 4G12 #05-515), Sigma (Actin #A2103) Santacruz (PDGF-B #sc7878) and BD Biosciences (CD31 antibody #553370).

### Immunoprecipitation

Cell extracts were prepared using Triton lysis buffer [20 mM Tris (pH 7.4), 1% Triton X-100, 10% glycerol, 137 mM NaCl, 2 mM EDTA, 25 mM b-glycerophosphate, 1 mM sodium orthovanadate, 1 mM phenylmethylsulfonyl fluoride, and 10 μg/mL of aprotinin and leupeptin] and incubated (16 hrs., 4° C.) with 10 μg control non-immune rabbit IgG (Santa Cruz) or with 10 μg rabbit antibodies to Acetyl Lysine (EMD Millipore). Immunecomplexes isolated using Protein G Sepharose were washed (five times) with lysis buffer.

### Statistical Analysis

All data are expressed as mean+SE and the numbers of independent experiments are indicated. Statistical comparisons were conducted between 2 groups by use of Student t test or Mann-Whitney U test as appropriate. Multiple groups were compared with either 1-way Kruskal-Wallis or ANOVA with a post hoc Tukey-Kramer multiple comparisons test as indicated in legends. A P value <0.05 was considered significant. All statistics were done using StatView version 5.0 (SAS Institute, Cary, N.C.) or GraphPad Prism version 5 (GraphPad Software, La Jolla, Calif.).

### Example 2—Loss of JNK3 Promotes Blood Flow Recovery and Angiogenesis after Hind Limb Ischemia (HLI)

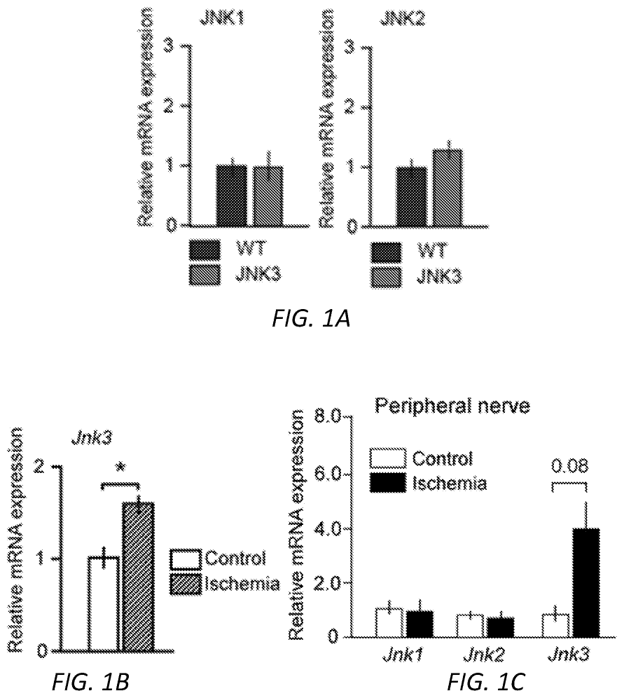

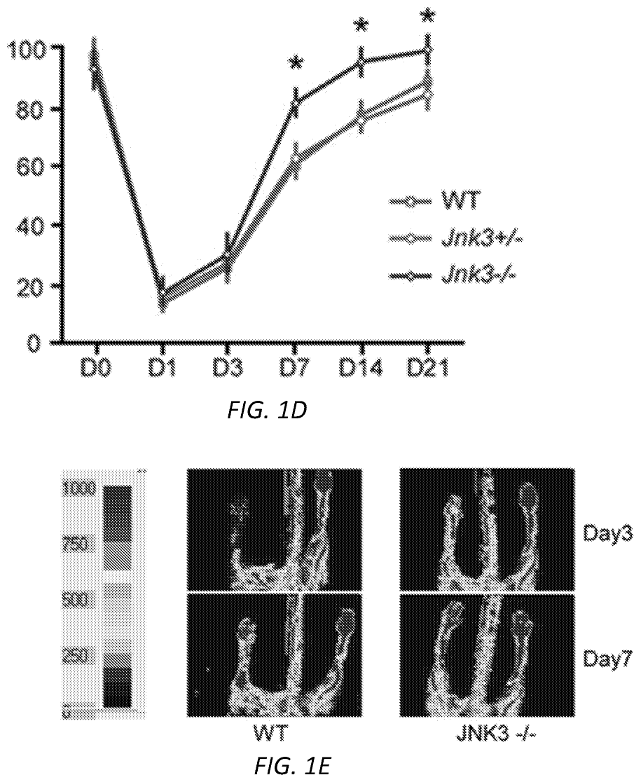

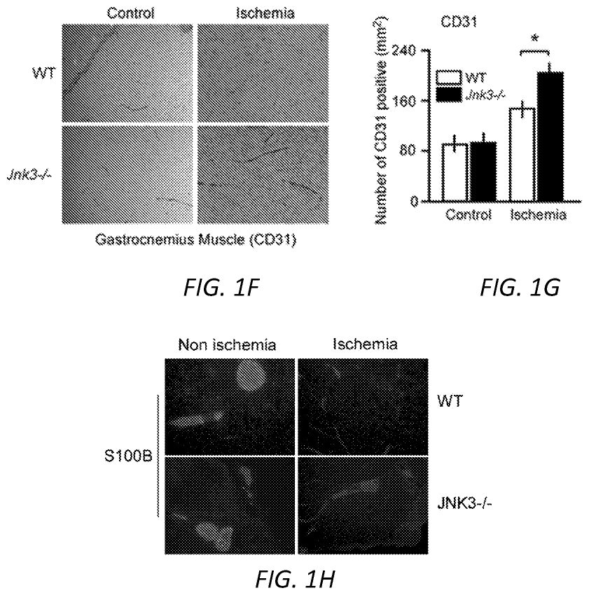

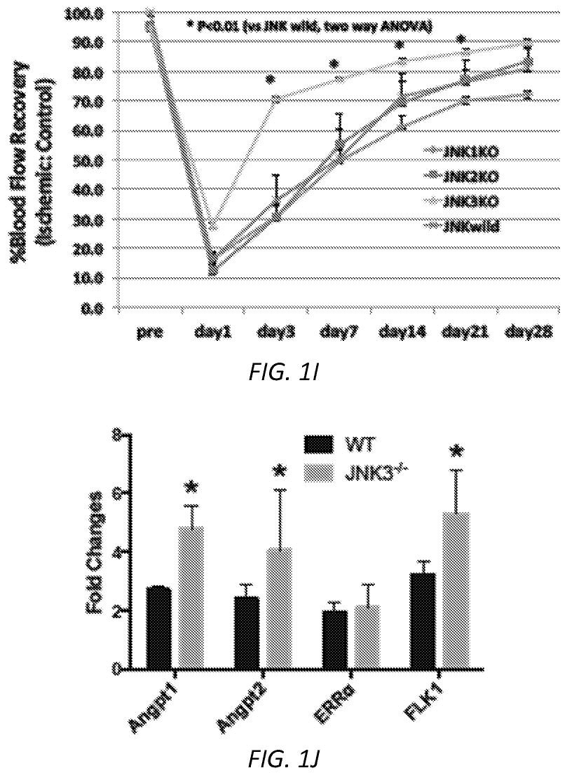

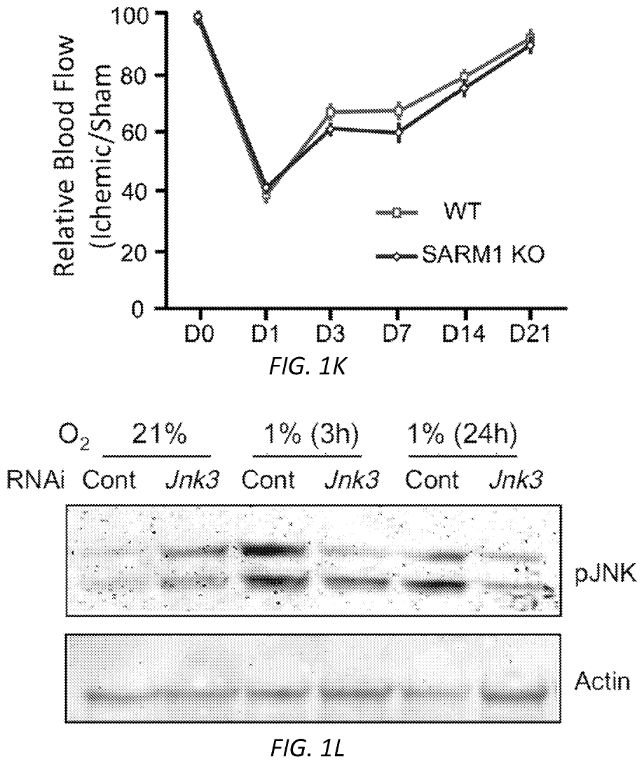

The tissue distribution of JNK1 and JNK2 are ubiquitous, but JNK3 is mainly expressed in neurons (Manning & Davis, Nat Rev Drug Discov 2003; 2: 554-56). In contrast to a previous report in bovine aorta (Pi et al., Proc Natl Acad Sci USA. 2009; 106:5675-5680), no detectable JNK3 was found in mouse lung or microvascular endothelium using two different primer sets (Ramo et al., Elife 2016; 5). These data suggest JNK3 cannot influence endothelial responses to tissue ischemia in a cell autonomous fashion. No compensatory upregulation of Jnk1 or Jnk2 mRNa in these mice was seen (FIG. 1A). Utilizing a femoral artery ligation model, a significant increase in JNK3 expression was observed in the gastrocnemius muscle of wild-type mice (FIG. 1B). This marked JNK3 increase was also observed in the peripheral nerves, wherein JNK3 is primarily expressed (Manning & Davis, Nat Rev Drug Discov 2003; 2: 554-565) (FIG. 1C). Interestingly, although they are expressed in neurons, no changes in JNK1 and JNK2 expression was observed in this model. These data suggested that ischemia regulates JNK3 expression in the peripheral nerves, which may in turn function in the neovascularization process.

To determine the role of JNK isoforms (JNK1, JNK2 and JNK3) in neovascularization during ischemia, a mouse model of peripheral arterial disease, hind limb ischemia (HLI) was utilized and blood flow recovery was quantified by laser Doppler perfusion imaging (LDPI) measurements in wild-type (WT), JNK1^{-/-}, JNK2^{-/-} and JNK3^{-/-} mice. No impact of JNK1 or JNK2 loss-of-function was observed (FIG. 1l). Compound loss of Jnk1 and Jnk2 together produced a developmental defect in collateral formation with no impact on post-natal blood flow recovery (Ramo et al., Elife 2016; 5). Unlike the JNK1 and JNK2-deficient mice where blood flow recovery was suppressed (Ramo et al., Elife 2016; 5), mice lacking the JNK3 isoform have accelerated recovery of blood flow in response to HLI (FIGS. 1D and 1E). Next, angiogenesis was examined in the GC using an endothelial marker, CD31. Staining with CD31 in the JNK3 null mice was significantly enhanced compared to WT after HLI (FIGS. 1F and 1G). In addition, compared to wild-type littermates, JNK3-null mice exhibited enhanced upregulation of Angiopoietin 1 (Angpt1), Angiopoietin 2 (Angpt2), and Fetal liver kinase-1 (Flk-1) (FIG. 1J).

As shown herein enhanced nerve integrity in Jnk3-null mice was seen in the hind limb ischemia model using the neuronal and glial marker, S100 (FIG. 1H). However, resistance to neural tissue death appears insufficient for the Jnk3′ phenotype as dSARAM^{-/-} mice, that are also resistant to injury-induced neural tissue death (Osterloh et al, Science. 2012:337:481-484) do not exhibit enhanced blood flow recovery in the hind limb ischemia model (FIG. 1K). These findings suggest a specific functional role of JNK3 in neural cells beyond simple prevention of cell death or apoptosis. These findings may explain enhanced ischemic blood flow recovery in Jnk3-null mice since, during development, peripheral nerves can influence key aspects of angiogenesis (Shireman, J Vasc Surg 2007; 45 Suppl A, A48-56) such as arterial patterning (Mukouyama et al., Cell. 2002; 109:693-705) and differentiation (Mukouyama, Development. 2005; 132:941-952). These effects are thought to occur through the generation of VEGF (Mukouyama, Development. 2005; 132:941-952; Fruttiger, Angiogenesis 2007; 10:77-88) and other ligand receptor pairs (e.g. semaphorins/plexin, netrin/unc5, slit/robo) common to both neuronal and vascular development (Larrivee et al., Circ Res. 2009; 104:428-441) that are now collectively called angioneurins (Zacchigna et al., Nat Rev Neurosci. 2008; 9:169-181). Indeed, severing the femoral nerve downregulates genes important for angiogenesis such as VEGF, Fltl, Tie2, NRP1 (Wagatsuma & Osawa, Acta Physiol (Oxf). 2006; 187:503-509) and inhibits ischemic blood flow recovery (Renault et al., Nerve Survival. Circ Res. 2013; 112: 762-770). These data suggest that JNK3 inhibition could enhance ischemic blood flow recovery via promotion of repair responses, including angiogenesis. Indeed, Jnk3 was activated by hypoxia (FIG. 1L) and Jnk3 suppression in neurons limited total JNK activation (FIG. 1L) and enhanced VEGF mRNA (FIG. 1M). Given that previous efforts to drive angiogenesis in PAD have failed (Simons, Circulation. 2005; 111: 1556-1566), these data suggest that previous efforts to focus only on vascular cells were incomplete and, perhaps, peripheral nervous tissue represents the key target to promote revascularization of ischemic limbs.

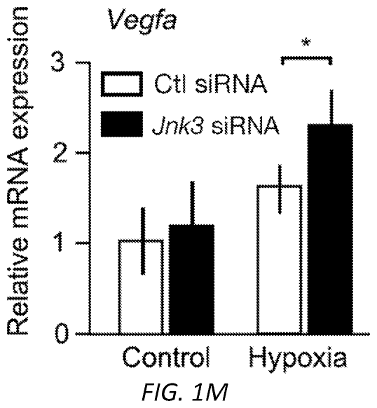

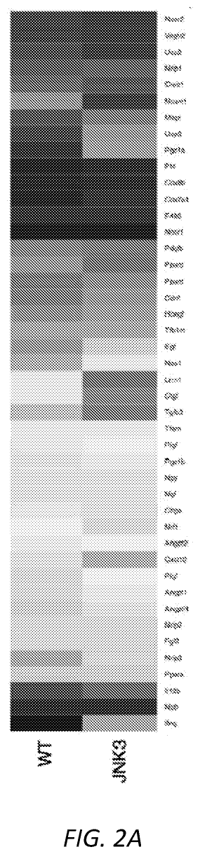

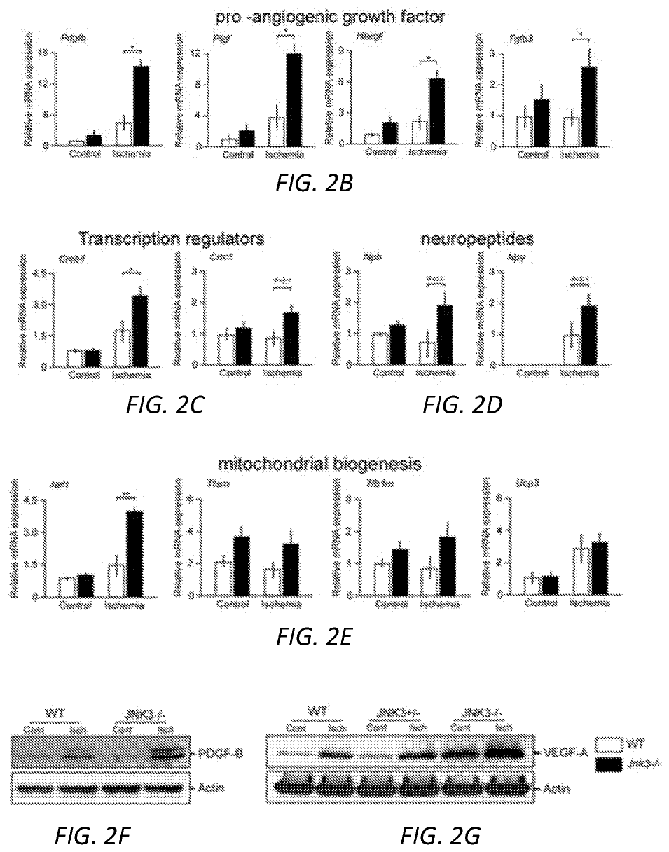

Since Jnk3 expression was not observed in endothelium, the data likely point to peripheral nerves as the key mediator(s) of JNK3-mediated enhanced angiogenesis and blood flow recovery. In order to ascertain the molecular mechanism(s) responsible for the accelerated blood flow recovery phenotype of JNK3 null mice, a micro-array analysis in gastrocnemius muscle from these mice was performed after hind limb ischemia. Notably, significant up-regulation of several pro-angiogenic growth factors (Eichler et al., Nat Rev Clin Oncol 2011; 8:344-356) in the JNK3 null muscle when compared to the WT was observed (FIG. 2A). The microarray data was confirmed by qRT-PCR for pro-angiogenic growth factors Pdgfb, Plgf Hbegfand Tgfb3 (FIG. 2B). In addition, markers of neuropeptide (Ekstrand et al., Proc Natl Acad Sci USA 2003; 100: 6033-6038) and mitochondrial biogenesis (Kluge et al., Circ Res 2013; 112: 1171-1188) were explored, both known to play a role in angiogenesis (Craige et al., Biochim Biophys Acta 2016; 1862: 1581-1586; Ekstrand et al., Proc Natl Acad Sci USA 2003; 100: 6033-6038) (FIGS. 2C-E). The gene expression results demonstrated that there is a significant upregulation of pro-angiogenic growth factors, but no change in neuropeptide and mitochondrial biogenesis markers (FIGS. 2B-E). The gene expression data was confirmed at the protein level as, there was a significant increase in protein expression of pro-angiogenic PDGF-B and VEGF-A in the JNK3-null gastrocnemius muscle after ischemia (FIGS. 2F and 2G). Together these data strongly indicate that pro-angiogenesis pathways are significantly activated in ischemic muscle from JNK3 null mice as compared to WT littermates. These data are highly novel as: i) they are one of the first animal models of accelerated blood flow recovery and; ii) only two other publications link the vasculature to Jnk3 with one suggesting that aortic JNK3 enhances endothelial proliferation (opposite of what is presently taught) (Pi et al., Proc Natl Acad Sci USA. 2009; 106:5675-5680), the other involving retinal pruning (Salvucci et al., Nat Commun. 2015; 6:6576); and neither touching upon ischemic responses.

### Example 3—JNK3 Regulates Pro-Angiogenic Factors Via Creb1 and the Sirtuin1/Creb1 Axis to Control Hind Limb Blood Flow Recovery Following Ischemia

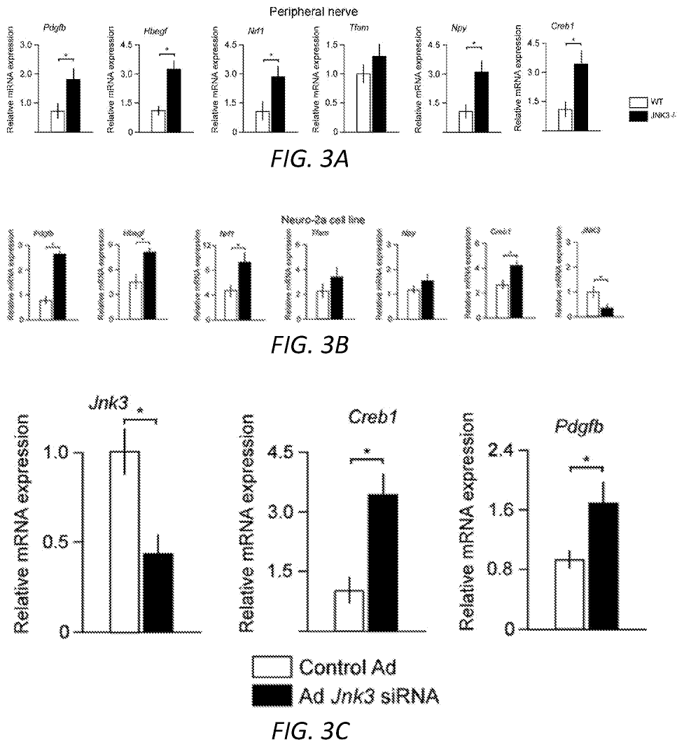

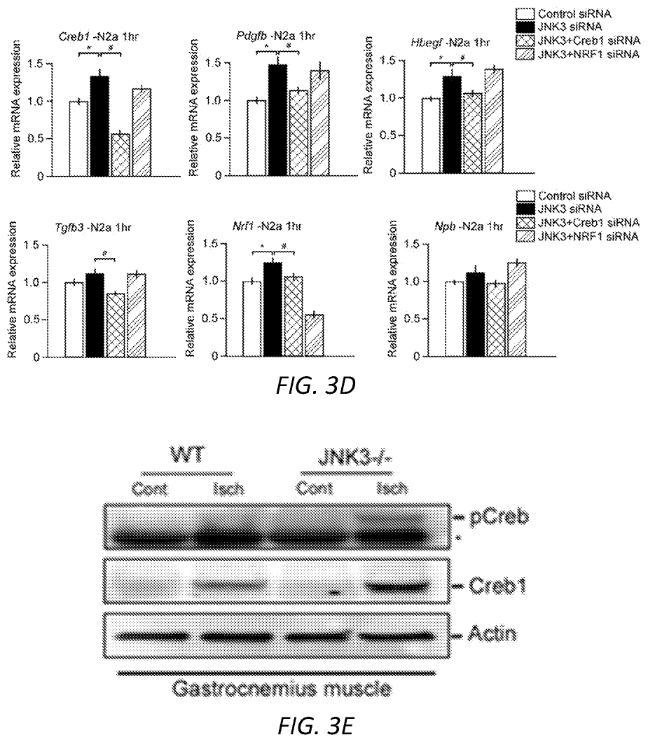

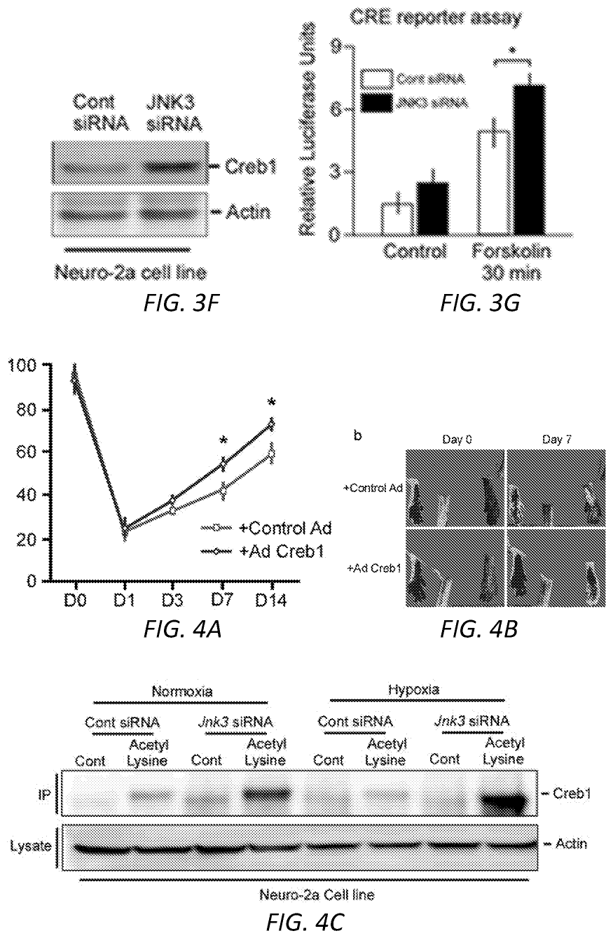

Increased endothelial marker expression (Zachary, Biochem Soc Trans 2003; 31: 1171-1177; Shireman, Vasc Surg 2007; 45 Suppl A, A48-56) was observed in the JNK3 null mice (FIGS. 1F and 1G), however, JNK3 expression was not detectable in endothelial cells isolated from WT mice. Although angiogenesis is conventionally associated with endothelial cell signaling (Zachary, Biochem Soc Trans 2003; 31: 1171-1177; Shireman, Vasc Surg 2007; 45 Suppl A, A48-56), there are multiple tissues involved in orchestrating the angiogenic response to ischemia (Segura et al., Trends Mol Med 2009; 15: 439-451). As JNK3 is highly expressed in neural tissue, the pro-angiogenic growth factor profile from peripheral nerves was examined in JNK3 null mice. The pro-angiogenic genes of Pdgfb, and Hbegf were indeed up-regulated in JNK3 null peripheral nerves as compared to those from WT littermates (FIG. 3A). The association between neural JNK3 expression and pro-angiogenic gene expression was confirmed using the neuroblastoma cell line Neuro-2a (N2a). In this cell line, JNK3 knockdown increased expression of pro-angiogenic factors Pdgfb and Hbegfj in hypoxic conditions (FIG. 3B).

In order to confirm that JNK3 loss in the peripheral nerves lead to increased pro-angiogenic growth factors in vivo, JNK3 knockdown was performed utilizing a gene painting technique that introduced small interfering RNA against JNK3 via an adenovirus delivered specifically to peripheral nerves. Delivering siRNA for JNK3 directly to the peripheral nerves shows an increase in Pdgfb (FIG. 3C), suggesting that neuron-targeted JNK3 inactivation could activate this pro-angiogenic pathway.

Two known transcriptional regulators of growth factors, Creb1 and Nrf1 were significantly upregulated in the JNK3 null mice based on the microarray and qRT-PCR data (FIGS. 2C and 2E). Interestingly, both have been implicated in several angiogenic pathways (Kluge et al., Circ Res 2013; 112: 1171-1188; Hamik et al., Arterioscler Thromb Vasc Biol 2006; 26: 1936-1947; Rhee et al., J Biol Chem 2015; 290: 26194-26203). To determine if these transcription factors are involved in mediating the pro-angiogenic effect of JNK3 knockdown in neurons, a double knockdown experiment with Jnk3 siRNA combined either with either Creb1 siRNA or Nrf1 siRNA was performed in the Neuro-2a cell line. Unlike Nrf1 knockdown, Creb1 knockdown was able to neutralize the upregulation of pro-angiogenic genes Pdgfb and Hbegffrom JNK3 knockdown in hypoxia (FIG. 3D). Together with the data in FIG. 2E demonstrating there was no change in the downstream targets (Gleyzer et al., Mol Cell Biol 2005; 25: 1354-1366; Choi et al., Ann N Y Acad Sci 2004; 1011: 69-77) ofNrfl (Tfam and Tfblm), suggests that Nrf1 is not responsible for mediating the pro-angiogenic effects in the JNK3 null mice.

Creb1 activation is largely regulated by phosphorylation of its serine residue 133 (Altarejos & Montminy, Nat Rev Mol Cell Biol 2011; 12: 141-151; Lonze & Ginty, Neuron 2002; 35: 605-623), and ischemic JNK3 null mice express substantially more phospho-Creb than WT (FIG. 3E). Furthermore, active Creb1 can up-regulate its own transcription (Liu et al., J Neurosci 31, 2011; 6871-6879), and indeed increased Creb1 expression at both the mRNA and protein levels was observed in JNK3-null mice (FIGS. 2C and 3E). These findings were further confirmed in the Neuro-2a cell line, in which both Creb1 mRNA and protein levels were found to be higher in JNK3 knockdown cells (FIGS. 3C, 3D and 3F).

Then the effect of JNK3 on Creb1 binding and transcriptional activity was examined, using a Creb1 response element (CRE) (Lonze & Ginty, Neuron 2002; 35: 605-623) luciferase reporter assay after Jnk3 knockdown in the Neuro-2a cell line. Suppressing JNK3 significantly increases the binding activity of Creb1 (FIG. 3G), suggesting that JNK3 suppression increases pro-angiogenic gene expression through Creb1 expression and activation.

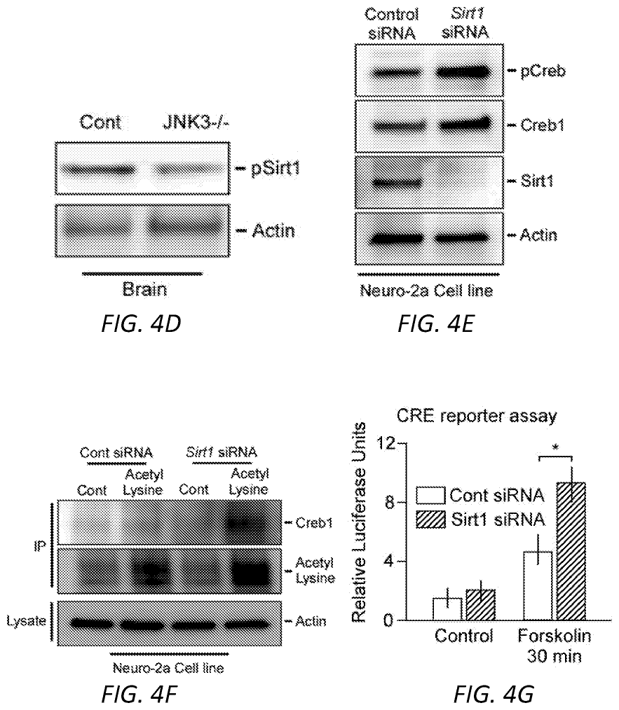

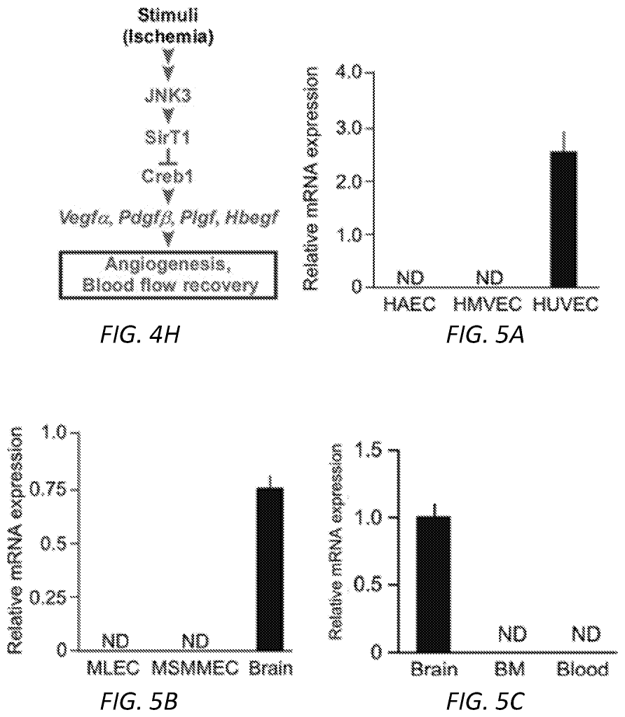

As shown in FIGS. 2C and 3, Creb1 expression was significantly increased in JNK3-deficient tissue and cells. In order to determine the specific role of Creb1 in ischemia-induced angiogenesis in vivo, Creb1 was over-expressed using adenovirus locally in the gastrocnemius muscle. Similar to the response in JNK3 null mice, the mice over-expressing Creb1 were able to recover blood flow significantly faster than the control mice following hind limb ischemia (FIGS. 4A and 4B). These data strongly implicate loss of JNK3 enhances blood flow recovery through Creb1 expression and activity in HLI.

Sirtuin 1 (SirT1) is a known Creb1 repressor (Monteserin-Garcia et al., FASEB J 2013; 27: 1561-1571) that acts to suppress the activity of Creb1, in part, by deacetylation (Monteserin-Garcia et al., FASEB J 2013; 27: 1561-1571). In the JNK3 knockdown cells, increased Creb1 acetylation is seen, which is further, enhanced in hypoxic conditions (FIG. 4C). It is known that SirT1 can be activated by another member of the JNK family, JNK1, through phorphorylation (Nasrin et al., PLoS One 2009; 4: e8414). Therefore, SirT1 phosphorylation was investigated in the JNK3 null mice; a significant decrease in SirT1 phosphorylation was observed (FIG. 4D). Furthermore, similar to what was observed with JNK3 knockdown, knockdown of SirT1 enhanced Creb1 acetylation (FIG. 4F). SirT1 can also suppress Creb1 by indirect dephosphorylation of its active site serine 133 (Choi et al., Ann N Y Acad Sci 2004; 1011: 69-77). With SirT1 knockdown, Creb1 phosphorylation at its active site serine 133 is increased (FIG. 4E). Furthermore, Creb1 was significantly more active after SirT1 knockdown, paralleling the effect seen with JNK3 knockdown (FIG. 4G). Together these data strongly suggest that JNK3 inhibits pro-angiogenic gene expression in ischemia via Creb1 suppression mediated by SirT1.

In conclusion, a novel inhibitory role for JNK3 on neovascularization was found. The increased blood flow recovery observed in JNK3^{-/-} mice is due to a previously unknown JNK3/Sirtuin1/Creb1 signaling axis. It was observed that JNK3 null mice have increased Creb1 expression and activity initiating transcription of growth factors. Indeed, Creb1 overexpression phenocopied loss of JNK3 as blood flow recovery was enhanced with Creb1 expression in HLI. Loss of JNK3 decreased SirT1 phosphorylation, enhancing acetylation of Creb1. SirT1 knockdown increased acetylation and phosphorylation of Creb1.

Creb1 is known to activate survival signaling in peripheral neurons (Liu et al., J Neurosci 31, 2011; 6871-6879), however the role of Creb1 in angiogenesis and blood flow recovery has not previously been described. Both decreasing JNK3 expression, as well as increasing the expression level of Creb1, can increase pro-angiogenic pathways and instigate vascular repair after ischemia. As current treatments for peripheral vascular diseases in humans are limited in effectiveness, this JNK3/Sirtuin1/Creb1 pathway serves as an intriguing and promising target for therapies aiming to improve the peripheral vasculature in diabetic and other effected patient populations.

### Example 4—Impact of Global Jnk3 Loss-of-Function on Tumor Angiogenesis

The hind limb ischemia model promotes blood flow recovery via vasodilation and recruitment of dormant collateral vessels present at birth, as well as via angiogenesis and arteriogenesis. Moreover, signaling from skeletal muscle (Arany et al., Nature. 2008; 451:1008-1012) and nerve tissues have important roles in hind limb blood flow recovery (Renault et al., Nerve Survival. Circ Res. 2013; 112: 762-770). In contrast, tumor angiogenesis depends solely on new vessel formation from tumor-derived stimuli. Thus, testing the implications of Jnk3 on tumor angiogenesis can provide mechanistic information. To determine the role of Jnk3 on tumor angiogenesis, a tumor model using PCR-tested mouse B16 melanoma cells (B16-F10; 1×10⁶cells) in 0.2 ml PBS that are inoculated into the left and right dorsal flanks of Jnk3^{-/-} mice and wild-type littermate controls (12/grp, 6 males, 6 females) is used. In this model, tumor growth depends upon angiogenesis (O'Reilly et al., Cell. 1997; 88:277-285) and tumors are easily recoverable for analysis due to pigmentation. The mice are monitored for weight, well-being, and tumor formation twice weekly and tumor diameter is measured with calipers over the skin of the intact animal. Animals are euthanized (before tumors reach 1 cm or ulcerate), and tumor tissue is harvested for gross photography as well as anatomy, histology, and immunohistochemistry. Immunostaining with CD31 and α-smooth muscle cell actin (SMA) is used for vascular morphology, density, and maturity. neurofilament-M immunostaining is used for nerve infiltration. Tumor mRNA is isolated for qRT-PCR and expression of HIF-1α and PGC-1α-driven genes are assessed (e.g., SDF-1, angipoetin-1, angplt-4, PDGF-β, PGC-la, and PGC-1β).

Since all the vascular tissue is derived from the host, these studies should indicate whether Jnk3^{-/-} vessels have enhanced angiogenesis capacity. The vessel morphology from CD31 staining of tumor tissue is indicative of whether or not Jnk3 status impacts the organization of capillaries, since vessel growth does not always ensure functional angiogenesis (Fraisl et al., Dev Cell. 2009; 16:167-179). If evidence for capillary disorganization is seen, additional experiments will be done with lysine fixable FITC-dextran injections to assess capillary leakage. If Jnk3 status confers any change in tumor size, immunoblots will examine tumor expression of VEGF, VEGFR2 (and its phosphorylation), PDGFR-β, TGF-βR1, EGFR, and Notch since these ligands and receptors have been implicated in angiogenesis and vascular permeability (Fraisl et al., Dev Cell. 2009; 16:167-179). The neurofilament-M staining will probe for nerve infiltration of the tumors since nerve-derived ligands have been implicated in tumor angiogenesis (Toda et al., Proc Natl Acad Sci USA. 2008; 105:13550-13555). If evidence of neurofilament-M staining is seen, the isolated tumor mRNA isolated above will be used to test for angioneurin expression.

### Example 5—Impact of Global Jnk3 Loss-of-Function on Cutaneous Wound Healing

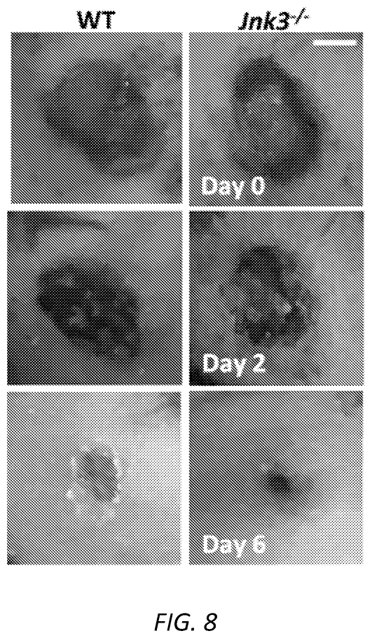

Impaired wound healing is a feature of vascular insufficiency and is responsible for significant morbidity and mortality. Wound healing is largely, though not completely, dependent upon angiogenesis and blood vessel supply. As shown herein, Jnk3-null mice have improved blood flow recovery with ischemia (FIG. 1) prompting speculation that JNK3 inhibition could speed wound healing. Accordingly, the impact of Jnk3 genotype on wound healing was tested by creating a sterile 6-mm wound on the dorsal flank skin with a biopsy punch (Miltex Inc, PA). Wound regions were photographed on a dissecting microscope (Leica) over 0-7 days (see FIG. 8).

The wound area is calculated from images using NIH ImageJ software. In separate animals, skin lesions are harvested after 4 days and are subjected to histology and immunohistochemistry.

### Example 6—Cell Type(s) Important for the Effect of Jnk3 Loss-of-Function

To determine the precise cell type(s) that are responsible for accelerated recovery of blood flow in response to hind limb ischemia, tissue-specific gene deletion was performed to determine which cell type(s) recapitulate the effect of the global Jnk3-null animal (Vernia et al., Elife 2016; 5: e10031). Theoretically, the findings in FIG. 1 could be due to the effects of JNK3 in nerve tissue, skeletal muscle or endothelium.

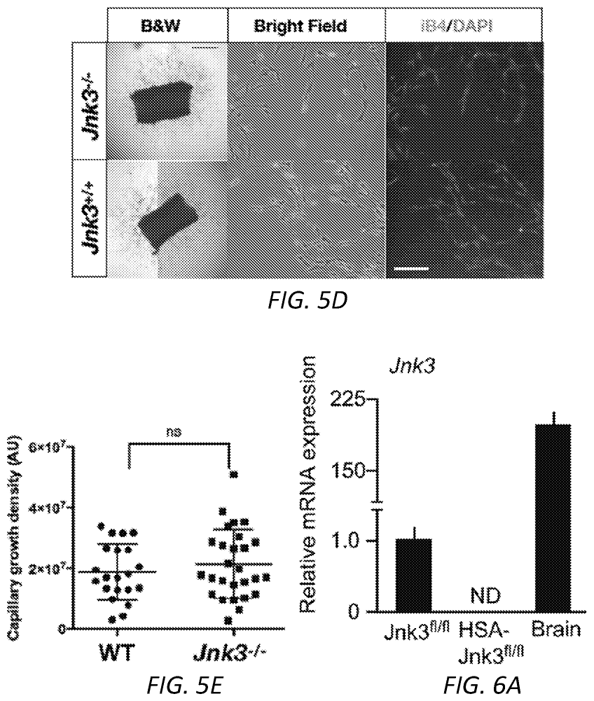

JNK3 expression and function in endothelium was examined. No Jnk3 expression was detected in human aortic endothelial cells (HAEC) or in human microvascular endothelial cells (HMVEC); some expression was noted in human umbilical vein endothelial cells (HUVEC), with this latter cell type not typically involved in capillary formation in vivo (FIGS. 5A-B). Similarly, there was no Jnk3 expression in mouse endothelium. Since endothelial progenitor cells are thought to come from bone marrow, bone marrow was examined. However, no Jnk3 expression was found in the blood or bone marrow (FIG. 5C). Consistent with these data, no impact of Jnk3 on aortic segment CD31+ capillary sprout formation was seen using established methods (Shimasaki et al., Circ Res. 2013; 113:891-901.) (FIGS. 5D-E). Collectively, these data indicate that the observations made in Jnk3′ mice cannot be explained by effects on the endothelium or bone marrow.

Floxed JNK3 mice were used to examine the impact of Jnk3 in: i) neural tissue or ii) skeletal muscle, on ischemic vascularization and wound healing.

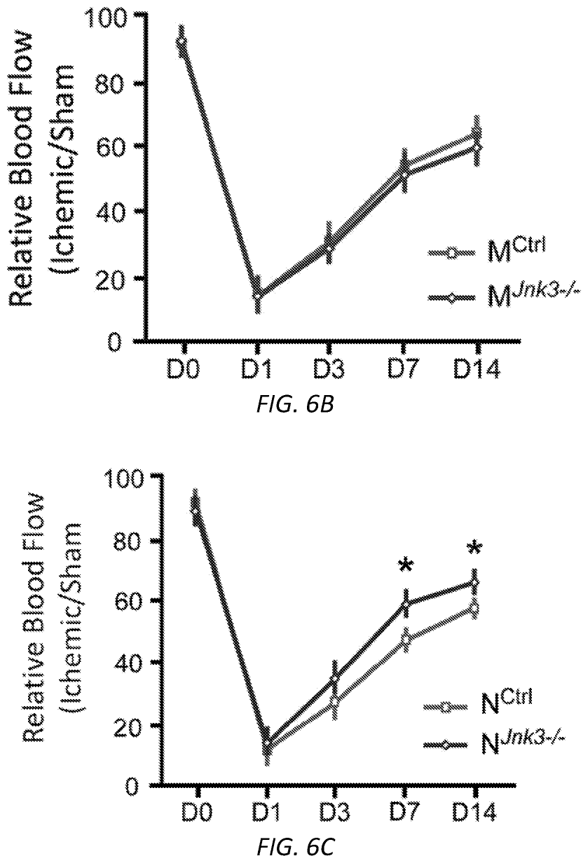

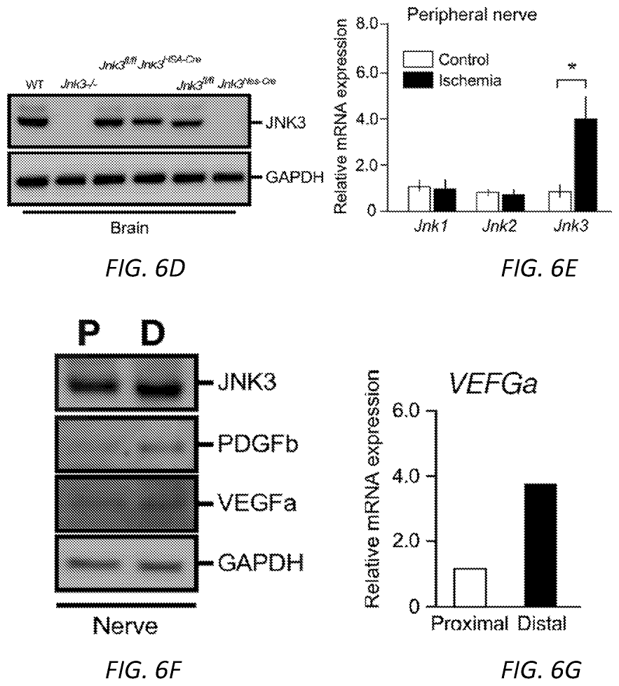

To examine muscle, conditional Jnk3^{fl/fl} mice were crossed with the human skeletal actin (HSA) Cre-driver line (Miniou et al., Nucleic Acids Res. 1999; 27:e27) to produce efficient skeletal muscle-specific gene excision. Muscle Jnk3 expression was 0.6% of that in nervous tissue (FIG. 6A), but was nevertheless completely excised by the HSA-Cre driver line (FIG. 6A) with no impact on nervous tissue Jnk3 (FIG. 6D). The resulting skeletal muscle Jnk3^{-/-} mice (M^{Jnk3-/-}) produced no impact in the hind limb ischemia model (FIG. 4B). In contrast, crossing Jnk3^{fl/fl} with the Nestin-Cre driver line that targets gene excision in the central and peripheral nervous system (including glia) (Tronche et al., Nat Genet. 1999; 23:99-103), produced Nestin-Jnk3^{-/-} mice (N^{Jnk3-/-}) that exhibited nervous tissue Jnk3 excision (FIG. 6D) and enhanced ischemic blood flow recovery compared to Nestin-Cre control mice (N^{Ctrl}; FIG. 6C). Thus, Jnk3 gene excision in nervous tissue, not muscle, conferred enhanced hind limb blood flow recovery. These data are consistent with reports that nerves and Schwann cells are important source(s) of VEGF (Mukouyama et al., Cell. 2002; 109:693-705; Mukouyama, Development. 2005; 132:941-952). Consistent with these data, ischemia-induced upregulation of Jnk3 was observed in mouse peripheral nerve tissue with no impact on Jnk1 or Jnk2 (FIG. 6E).

To probe the human relevance of these findings, JNK3 and VEGFa expression was investigated in freshly obtained human lower-limb tissue derived from patients with PAD undergoing amputation for vascular insufficiency. The site of amputation was in proximal healthy tissue (to aid healing), whereas distal tissue is typically ischemic. Strikingly, upregulation of JNK3, VEGF and PDGF was observed in distal ischemic nerve tissue compared to the expression levels in relatively healthy proximal tissue (FIGS. 6F-G). These data indicated that JNK3 was upregulated in human peripheral nervous tissue and that considerable VEGF was derived from this tissue.

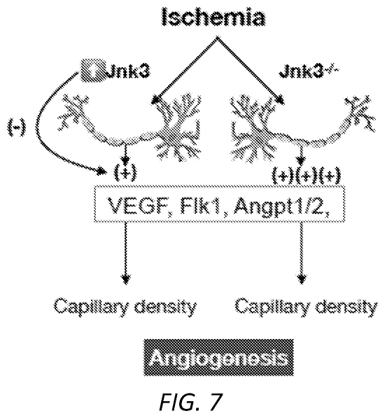

Limb ischemia, such as might be observed in human peripheral arterial disease, is associated with upregulation of stress-responsive pathways such as HIF-la and JNK, including the JNK3 isoform in muscle and neurons. The present data indicated that Jnk3^{-/-} mice (but not Jnk1^{-/-} or Jnk2^{-/-} mice) have enhanced ischemic blood flow recovery driven by increased angiogenesis responses (FIG. 1). Thus JNK3 is a key determinant of ischemia-driven angiogenesis as outlined in the central paradigm in FIG. 7. Since Jnk3 expression was not observed in endothelium, the data point to peripheral nerves as the key mediator(s) of JNK3-mediated enhanced angiogenesis and blood flow recovery.

Collectively, these data suggest JNK3 had a restraining effect on gene upregulation necessary for ischemia-induced angiogenesis, likely in peripheral nervous system. Peripheral nervous tissue includes axons and glia, which is composed of myelinating and non-myelinating Schwann cells. Myelinating and non-myelinating Schwann cells are known to be important in coordinating the response to nerve injury (Sulaiman et al., Exp Neurol. 2002; 176:342-354; Wong et al., Neural Regen Res. 2017; 12:518-524).

### Example 7—Egr1 Regulation Explains the Jnk3^{-/-} Phenotype

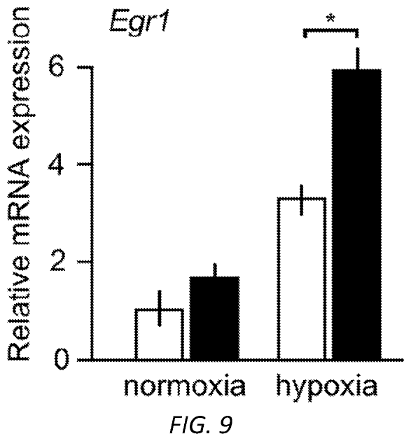

In considering the possibility that Hif1α-mediated gene transcription may not completely explain the upregulation of angiogenesis-related genes, a microarray was performed to compare Neuro-2a (N2a) cells exposed to hypoxia with or without Jnk3 siRNA. Hypoxia-induced upregulation of the transcription factor Egr1 with Jnk3 loss-of-function was confirmed by qRT-PCR (FIG. 9). This finding was of particular interest as EGRI has been implicated in ischemic stress responses (Yan et al., Nat Med. 2000; 6:1355-1361.), vascular repair (Khachigian et al., Science. 1996; 271:1427-1431) and angiogenesis (Fahmy et al., Nat Med. 2003; 9:1026-1032), Indeed, EGRI can induce many of the same genes observed in this system including Vegfα, Pdgfb, Hif1α, and TGFβ, but appears to do so via a Hif1α-independent mechanism that involves PKC and ERK pathways (Yan et al., J Biol Chem. 1999; 274:15030-15040), and activated JNK can inhibit ERK (Lei et al., Mol Cell Biol. 2002; 22:4929-4942). Combined upregulation of EGRI and Hif1α can lead to cooperative Vegfa transcriptional upregulation in a Hif1α-independent manner (Shimoyamada et al., J Pathol. 2010; 177:70-83). Finally, mice lacking Egr1 have impaired wound healing and tissue repair (Braddock, Ann Med. 2001; 33:313-318). Collectively, these data suggest Jnk3-mediated ERK inhibition and Jnk3^{-/-}-mediated upregulation of Egr1-dependent transcription could, at least in part, explain the Jnk3^{-/-} phenotype regarding hind limb ischemia and wound healing.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating angiogenesis-related disorders in a subject, the method comprising administering to the subject a therapeutically effective amount of an agent that inhibits JNK3 expression and/or activity.

2. The method of claim 1, wherein the agent is a small molecule or an inhibitory nucleic acid.

3. The method of claim 2, wherein the inhibitory nucleic acid is an antisense molecule, a small interfering RNA, or a small hairpin RNA which are specific for a nucleic acid encoding SEQ ID NO: 1.

4. The method of claim 2, wherein the inhibitory nucleic acid is a nucleic acid comprising a sequence that is complementary to a contiguous sequence of at least 5 nucleotides present in JNK3.

5. The method of claim 1, wherein the agent is a peptide or a peptide-inhibitor.

6. The method of claim 5, wherein the peptide inhibitor is a peptide JNK3 inhibitor.

7. The method of claim 1, wherein the subject is human.

8. The method of claim 1, wherein the subject has, or is at risk of having an angiogenesis-related disorder.

9. The method of claim 1, wherein the subject has peripheral arterial disease (PAD), macular degeneration, retinopathy, stroke, diabetic limb ulcers, diabetic neuropathy, age-related blindness, chronic wounds, pressure ulcers, Alzheimer's disease, myocardial ischemia, cerebral ischemia, hepatic ischemia, limb ischemia, pulmonary ischemia, renal ischemia, testicular ischemia, intestinal type ischemia, or any organ ischemia.
